(19) United States
(12) Patent Application Publication
(10) Pub. No.: US 2007/0292943 A1
(43) Pub. Date: Dec. 20, 2007

(54) MOLECULAR SHUTTLE DEVICES

(75) Inventors: Joseph M. JACOBSON, Newton, MA (US); David W. MOSLEY, Cambridge, MA (US); Brian CHOW, Cambridge, MA (US)

Correspondence Address: NORMA E HENDERSON;HENDERSON PATENT LAW, 13 JEFFERSON DR, LONDONDERRY, NH, 03053, US

(73) Assignee: MASSACHUSETTS INSTITUTE OF TECHNOLOGY, Cambridge, MA (US)

(21) Appl. No.: 11/733,187

(22) Filed: Apr. 9, 2007

Related U.S. Application Data
(62) Division of application No. 10/622,126, filed on Jul. 17, 2003, now abandoned.
(60) Provisional application No. 60/396,356, filed on Jul. 17, 2002.

(51) Int. Cl. C12N 15/63

(52) U.S. Cl. 435/320.1

(57) ABSTRACT

A molecular shuttle device comprises two or more molecular subunits connected to form a molecular chain, with the subunits defining binding positions along the molecular chain, and a shuttle capable of binding at each of the binding positions, wherein the shuttle moves between binding positions in response to one or more input signals which interact with the molecular subunits. In some embodiments the one or more input signals comprise an input signal sequence that interacts with the molecular chain and the shuttle moves between binding positions in response to the input sequence.

### RELATED APPLICATIONS

This application is a divisional application of U.S. patent application Ser. No. 10/622,126, filed Jul. 17, 2005, which claims the benefit of U.S. provisional patent application Ser. No. 60/396,356, filed Jul. 17, 2002, the entire disclosures of which are incorporated herein by reference.

### TECHNICAL FIELD

This invention generally relates to molecular shuttle devices and, in particular, this invention relates to molecular shuttle devices for the construction of molecular switches, molecular assemblies, and molecular memory devices and methods for producing the same.

### BACKGROUND

Molecular “shuttles” have been studied in recent years, but a molecular assembly line has yet to be constructed. Typically, these shuttles move an individual physical object, such as nanoparticles or molecules, back-and-forth between two positions in the manner of a switch. A uni-directional multi-position molecular shuttle has also been studied. Molecular logic switching devices and molecular memory devices have been proposed. Many of these devices, however, utilize components at the molecular scale that are placed on a solid substrate, such as nanoparticles, self-assembled monolayers, and individual atoms. Such an approach precludes the possibility of incorporating solution-based devices. In addition, even though the devices are based on molecular components, the devices are not truly molecular-scale because they require (non-molecular) electrodes to operate. The attainable size of the devices is therefore limited to the minimum electrode size attainable, for example, by lithographic techniques.

### SUMMARY

The invention features molecular scale devices that may be used as bi-stable switches, bi-directional molecular assembly lines, and molecular memory devices. The devices are composed of molecular subunits and shuttles that move between the subunits. As used herein the term “molecular” is used to indicate that the components which make us the devices are truly molecular-scale components. These molecular components are characterized by the fact they may be produced by chemical means, rather than by lithographic means.

One aspect of the invention provides a molecular shuttle device that includes two or more molecular subunits connected to form a molecular chain, the subunits defining binding positions along the molecular chain, and at least one shuttle capable of translating along the chain by means of sequentially breaking and forming bonds between the subunits and the shuttle. Thus, in one embodiment, the shuttle is a translatable ligand capable of translating along the chain by means of sequentially breaking and forming bonds between the subunits and the shuttle. As discussed in greater detail below, the breaking or formation of the bonds may be mediated by external input signals.

One aspect of the invention provides molecular devices and systems used to move shuttles along a molecular chain composed of connected molecular subunits which define binding positions for the shuttle along the chain. The shuttles move from one binding position to another in response to one or more input signals. The shuttles are capable of binding to any binding position along the chain and may move bi-directionally along the chain. That is, the shuttle may move forward or backward along the chain in response to an appropriate input signal or series of input signals.

The devices are made from a chain of connected molecular subunits which define binding positions along the chain. Shuttles capable of binding to the subunits move between adjacent binding positions along the chain in response to a series of input signals which selectively make or break bonds between the shuttle and the subunits. In this sense, the input signals act as connectors between device components. The shuttle may be any physical object of a suitable size to move along a molecular chain. In some devices, the shuttle will have micro-scale or nano-scale dimensions, although the shuttles are not limited to objects in these size ranges. In some embodiments, the shuttle desirably composed of a bead, such as a glass bead. Thus, a “shuttle” refers to an object to be moved between bound positions along the polymer chain in response to one or more input signals.

The devices are particularly useful as molecular assembly lines. A molecular assembly line is a device that assembles a molecule by bringing the molecular building blocks of a growing molecule into sufficiently close proximity to the growing molecule to allow the building blocks to react with the growing molecule, continuing the growth process. In the assembly lines provided herein, an assembly molecule may be transported by a shuttle to any binding position along a molecular chain that brings the growing molecule into sufficiently close proximity to a building block disposed along the chain to allow the building block to react with the assembly molecule. As used herein, simply refers to a base molecule to which molecular building blocks are added. Thus, the new molecule that results after each building block addition is redefined as the assembly molecule. Because the shuttles are capable of occupying any binding position along the molecular chain, the assembly lines provide a great deal of flexibility with respect to the order of addition of the building blocks.

Each of the subunits along a chain is capable of receiving an input signal that either produces a binding interaction that connects the subunit to the shuttle or terminates a binding interaction, disconnecting the subunit from the shuttle. In some instances, there is no binding affinity between the shuttle and the subunits in the absence of an input signal. In these systems, an input signal must be present in order for the shuttle to be connected to the molecular chain. Input signals that create bonds between the shuttle and the subunits are usefully employed in such systems to move the subunits from position to position. In other systems, the shuttle binds to the subunits even in the absence of an input signal. Input signals that break bonds between the shuttle and the subunits are usefully employed in such systems to move the subunits from position to position. In one exemplary embodiment, a device may be exposed simultaneously to two or more input signals which “turn off” the binding interactions between the shuttle and multiple subunits, such that the shuttle diffuses to and binds to a subunit for which the binding affinity remains “turned on.”

The input signals for neighboring subunits along a chain are desirably resolvable. An input signal is considered resolvable if it terminates binding between a given subunit in a molecular chain and a shuttle without terminating binding between the shuttle and the neighboring subunits along the chain or if it results in binding between a given subunit in a molecular chain and a shuttle without resulting in binding between the shuttle and the neighboring subunits along the chain. Thus, each of the subunits along the chain may be remotely and individually addressed with respect to its neighboring subunits. It follows that by exposing the molecular chain to an appropriate set of input signals, either sequentially or simultaneously, the shuttle may be selectively moved from one position to the next along the chain. This motion may include both unidirectional and bi-directional motion.

Another aspect of the invention provides molecular memory devices made from assemblies of connected molecular subunits.

The molecular devices provided herein are assembled and operate in solution rather than being supported on substrates like conventional solid state devices. As such, the molecular devices provided herein are free shuttle and assemble molecules in solution, rather than on a two dimensional surface. Suitable solvents in which the devices may operate include any solvent in which the molecular chains are non-interacting within the limit of diffusion. For example, water is not a suitable solvent for an organic system, as the chains will aggregate due to hydrophobic effects. This is advantageous because solution-based remote input devices are more versatile than the wired inputs of two-dimensional silicon-based architectures. Furthermore, remotely addressable systems can potentially have a greater device density than two-dimensional very large scale integrated (VLSI) based architectures. At present, silicon-based computer chips contain approximately one trillion logic elements, and the maximum memory density is 1 gigabyte (GB) per square inch. With solution-based switches, one trillion logic elements can be contained in one picoliter (pL) (1×10⁻¹² L) of 1M-solution. Likewise, 75 femtoliters (fL) (75×10⁻¹⁵ L) of 1M solution can hold as much memory as one five-inch wafer in a computer hard drive.

### BRIEF DESCRIPTION OF THE DRAWINGS

FIG. 1. Schematic illustration of a general approach toward untethered molecular shuttle devices.

FIG. 2. Schematic illustration of a molecular shuttle device with a tethered shuttle.

FIG. 3. Bi-stable switch/molecular memory with simultaneous and in situ read-out mechanism.

FIG. 4. Schematic illustration of memory construction.

FIG. 5. Schematic illustration of method for “writing” molecular memory.

FIG. 6. Schematic illustration of a tethered molecular assembly.

FIG. 7. Tethered molecular assembly line using a cyclodextrin polymer and optical inputs.

FIG. 8. Tethered molecular assembly line using photochromic zwitterions as subunits.

FIG. 9. Bi-stable molecular switch with spectroscopic readouts.

FIG. 10. Tethered molecular assembly line with chemical inputs.

FIG. 11 shows an idealized DNA assembly scheme using the proposed molecular assembly line. Top: Support is positioned over an adenine residue. Middle: Polymerase is added and the nucleotide is now on the solid-support while a diphosphate is left on the polymer backbone. Bottom: The support is positioned over a cytosine residue and the enzymatic coupling step occurs again.

FIG. 12. a) shows a size distribution of cobalt nanoparticles obtained from TEM micrographs, with d=2.8±0.7 nm. b) TEM micrograph of cobalt nanoparticles. Scale bar=10 nm. c) TEM micrograph of 4 nm single crystalline cobalt nanoparticle.

FIG. 13. a) Idealized structure (left) and increased fluorescence upon melting (right) of the DNA hairpin-loop. b) Coupling and purification strategy of covalent attachment of cobalt to DNA hairpin-loop.

FIG. 14. Perspective view of the spectrometer compatible RF apparatus. b) Top-down view as designed in Rhinoceros 3D modeling program. c) Schematic of cell holder with cooling as integrated into spectrometer.

FIG. 15. Global temperature of 1×PBS buffered water under 300 MHz irradiation for one hour.

FIG. 16. Melting curves of DNA with no cobalt nanoparticle (black circle), non-covalent cobalt nanoparticle (green triangle), and covalently attached cobalt nanoparticle (red square).

FIG. 17. Selective hybridization of DNA hairpin-loop with covalently attached 2.8 nm cobalt nanoparticle antenna. a) Fluorescence spectra of DNA/nanoparticle assembly in a 300 MHz field for 200 seconds (red). b) DNA hairpin-loop with no antennae in 300 MHz field under the same conditions as in (a).

FIG. 18. a) Maximum FAM emission vs. frequency from DNA/nanoparticle assembly. The Gaussian fit shows that RF coupling into the nanoparticle antennae is resonant, centered at 306 MHz with FWHM=40 MHZ (or σ=20 MHz). b) Reflected power spectrum of the coil; the response is not related to the field strength of the coil.

FIG. 19. a) Synthetic scheme for 5-(p-tolyl)-5-cyano-4,4-dimethyl-2-pentanone (X). b) Synthetic scheme for 4-(p-tolyl)-4-cyano-3,3-dimethyl-N,N-dimethylbutaneamide (Y).

FIG. 20. a) and b) HPLC chromatographs of (X) and (Y) after 48 hours UV irradiation, respectively.

FIG. 21. a) HPLC chromatographs of compound (X). Left, after 48 hours irradiation at λ>290 nm only. Right, after 48 hours stirring in the presence of LiHMDS with and without UV irradiation. b) HPLC chromatographs of compound (Y). Left, after 48 hours irradiation at λ=215 nm only. Right, after 48 hours stirring in the presence of LiHMDS with and without UV irradiation.

FIG. 22. Three-bit shift register memory, in which a physical bead represents a “1,” and the absence of a bead represents a “0.” Here, a memory of “1-0-1” is written, stored, and read. The bits are each shifter simultaneously by the same 1-2-3 pulse.

### DETAILED DESCRIPTION

Molecular devices and systems used to move shuttles along a molecular chain are provided. The devices are composed of chains of two or more connected molecular subunits which define binding positions along the chain. The shuttle is capable of binding at any binding position on the chain. The binding positions may be defined by individual subunits or by neighboring pairs of subunits along the chain. Each subunit is adapted to interact, directly or indirectly, with at least one input signal. The input signals act as connectors by interacting with the subunits in a manner that causes bonds between the subunit and the shuttle to form or break. However, neighboring subunits in a molecular chain are not adapted to respond to the same input signals. Thus, the input signals are selective, in that they only affect the bonding of subunits that are adapted to interact with them. The input signals are also resolvable, where a resolvable input signal is defined, for the purposes of this disclosure, as an input signal that is capable of making or breaking bonds between a shuttle and a subunit without making or breaking bonds between the shuttle and the neighboring subunits. In order to move a shuttle from position to position along the chain, the chain is exposed to a series of one or more resolvable input signals which selectively make and break bonds between the shuttle and the subunits in such a way that the shuttle moves from binding position to binding position along the chain in response to the input signals. Exposure to the input signals may take place simultaneously or sequentially.

Together the subunits, shuttle and input signals form a system. The shuttle may bind to each subunit individually as it moves from position to position along a molecular chain. In this embodiment, an existing bond between a shuttle and a subunit is broken and a new bond between the shuttle and a new subunit is formed, upon exposure to one or more appropriate input signals. During the transfer of the shuttle from one subunit to the next, the shuttle becomes disconnected from the molecular chain and moves from one position to the next by diffusion in solution. Alternatively, the shuttle may be bound between adjacent pairs of subunits as it moves along the chain. In this embodiment, the shuttle is “tethered” between adjacent subunit pairs. A shuttle is tethered between two subunit pairs if it is simultaneously bound to both subunits when it is disposed between them. Upon exposure to one or more appropriate input signals, an existing bonds between the shuttle and a subunit is broken and a new bond is formed between the shuttle and a new subunit, such that the shuttle moves from a first position between a first pair of adjacent subunits to a second position between a second pair of adjacent subunits, wherein the first and second subunit pairs have one subunit in common. Thus, in a “tethered” system, the shuttle always remains bound to the molecular chain, even as it shifts position. In an “untethered” system, an input signal frees the shuttle from the chain entirely.

The bonds between the shuttle to the subunits may be formed by physical, chemical or electromagnetic interactions. For example, the bonds may be intramolecular covalent bonds, or intermolecular bonds, such as Van der Waals forces, dipole interactions, hydrogen bonds, and electrostatic attraction/repulsion.

The input signals may be any input capable of making or breaking bonds between the shuttle and a subunit. The input signals may be physical inputs or chemical inputs. Examples of physical inputs include, but are not limited to magnetic inputs and electric inputs. Sources of physical input signals include, but are not limited to electric fields, magnetic fields, and electromagnetic radiation, including ultraviolet radiation, infrared radiation and visible light. Examples of chemical inputs include, but are not limited to compounds that change the chemical environment of the system. For example, compounds which alter the pH of the solution in which the devices operate may be used as chemical inputs. Whether physical or chemical inputs are employed and the nature of the inputs will depend on the nature and binding characteristics of the device. The input signals are preferably non-destructive, in the sense that they are able to alter the binding affinity between a shuttle and a subunit without cleaving other bonds in the system, such as bonds in the building blocks or in the growing molecules which the devices are being used to assemble. The input signals may produce global affects on the solution. This is often the case with chemical inputs. Alternatively, the input signal may produce local affects, limited to the molecular components of the devices. This is more common with a physical input such as electromagnetic radiation.

One specific example of an input signal suitable for some devices is an alternating radio frequency magnetic field (AC-RFMF). When AC-RFMFs are used as the input signals for a molecular assembly line, the input may be completely non-destructive in that it will not significantly affect the bonds in the molecular assemblies. AC-RFMF is a remote input that creates a local change, without affecting the solution. Optical inputs may also desirably be employed in some systems. An optical input signal is not destructive so long as the assembled species do not contain chemical moieties that are photo-cleaveable by that input. Furthermore, optical inputs are remote systems that only affect local environments. Chemical inputs, such as compounds that change the pH of a system may also be suitable for some applications. Such chemical inputs are not destructive so long as the assembly process is independent of pH conditions. However, chemical inputs may be limited because the input is global and thus, individual molecules cannot be addressed in solution.

The input signals may interact with the subunits, either directly or indirectly, through different mechanisms which affect the binding interactions between the subunits and the shuttles. In some instances, the input signals interact with the subunits in such a way that the binding interactions between the subunits and the shuttles are “turned off” disconnecting the shuttle from the subunit. For example, the input signals, such as electric and magnetic input signals, may result in the local heating of the subunit, causing non-covalent intermolecular bonds between the subunit and the shuttle to break, breaking the connection between the shuttle and the subunit. In other embodiments, input signals, may cleave (e.g., photocleave) covalent bonds between the subunits and the shuttle. For example, a carbonyl bond can be cleaved by a laser pulse to free the shuttle from a molecular chain.

In other embodiments, the input signals interact with the subunits in such a way that the binding interactions between the subunits and the shuttles are “turned on,” connecting the shuttle to a subunit. For example, the input signals may result in local heating of the subunit, kinetically biasing the system in favor of non-covalent bond formation between the subunit and the shuttle. In other embodiments, local heating caused by the input signals may turn on biomolecular activity, resulting in binding between a shuttle and a biomolecular subunit.

Other mechanisms through which input signals either turn on or off binding interactions include the following. Input signals may cause a subunit to undergo a conformational change, such as a cis-trans isomerization, that turn binding interactions between the shuttle and the subunit on or off. Alternatively, the input signals may convert a subunit from a ionized to a deionized state or vice versa, turning ionic binding interactions between the subunit and an ionic shuttle on or off. In other embodiments, input signals may be used to induce electron transfer reactions which may turn binding interactions on and off. An example of a system that is affected by electron transfer is described by Winkler, et al. in Biophysical Chemistry, 54, 199-209 (1995), the entire disclosure of which is incorporated herein by reference. In still other embodiments, binding interactions may be affected by input signal-induced changes in spin polarization states. In other systems, chemical inputs may enhance or destroy chemical interactions, including biochemical interactions, between the shuttle and the subunits by altering the chemical environment of the system.

The input signals for neighboring subunits in a molecular chain are resolvable. Therefore the neighboring subunits do not interact with the same input signals. Sometimes this selective interaction is the result of chemical differences between the subunits. In other instances, identical subunits are attached to antenna adapted to receive input signals, wherein the antenna on neighboring input signals are different and are adapted to receive different input signals. For the purposes of this disclosure, an “antenna” refers to a chemical species that receives an input signal and produces a response in the subunit to which it is attached. Thus, a subunit may interact indirectly with an input signal through an antenna.

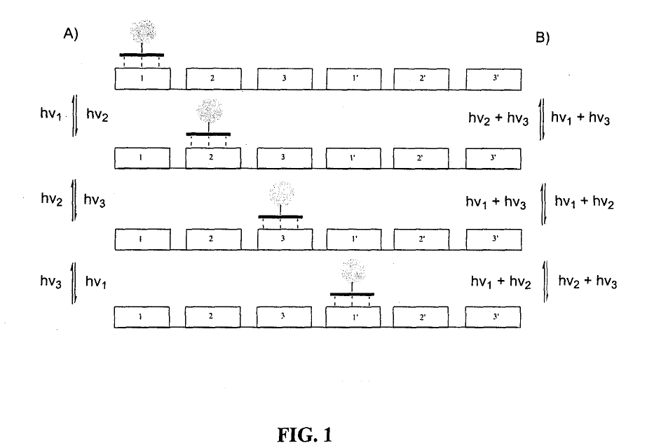

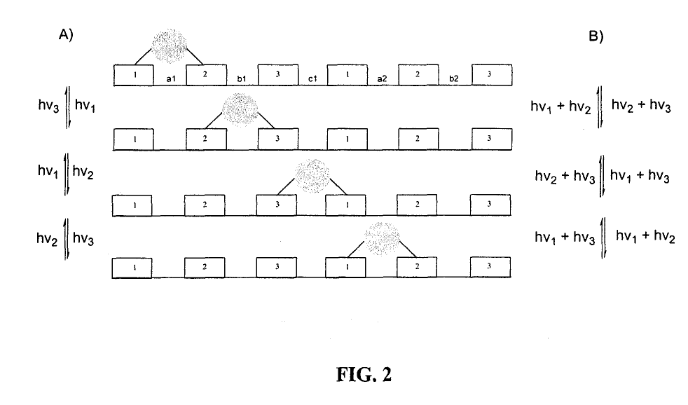

By exposing a chain of subunits having a shuttle bound thereto to an appropriate series of input signal sequence, the shuttle may be induced to move from position to positions along the chain. Depending on the exact sequence of the input signals, this motion may be uni- or bi-directional. The shuttle may also change direction as it travels along a chain. This is illustrated in FIGS. 1 and 2 which show schematic diagrams of a shuttle stepping along the subunits of a molecular chain in response to a sequence of input signals.

FIG. 1 illustrates a general approach toward untethered molecular shuttle devices, such as switches and molecular assembly lines. In FIG. 1A, input frequencies (i.e., signals) selectively “turn on” attractive intermolecular forces between the shuttle and successive subunits along the chain. In the absence of an input, there is no binding affinity between the shuttle and any of the subunits. Therefore, applying a specific input frequency moves the shuttle to the nearest affected position. However, the utility of such a system is limited because it is not bi-stable, since the input must be present in order for the shuttle to remain bound. The system is also limited by diffusion of the shuttle into solution. For example, if the shuttle is at position 1, it is biased toward position 2 by first breaking the non-covalent bonding between the shuttle and subunit 1. The shuttle then diffuses into solution. Applying the input frequency ν₂ (corresponding to the quantized energy value hν₂) increases the binding affinity between the shuttle and position 2, and thus moves of the shuttle toward position 2. Furthermore, diffusion in solution biases the shuttle toward position 2 over the more distant position 2′, where the binding affinity is equal to that in position 2.

As shown in FIG. 1A, the shuttle can then be moved from position 2 back to position 1 or moved forward to position 3 by applying input signals of frequency ν₁ or ν₃, respectively. The system is therefore bi-directional. Furthermore, the shuttle can be moved between any two positions along the chain by applying a series of input signals having the correct pulse/frequency sequence.

In FIG. 1B, the input frequencies selectively break the non-covalent bonds between the subunit and the shuttle. In the absence of any input, the shuttle has an equal binding affinity for any subunit. The system in FIG. 1B operates in a manner opposite that shown in FIG. 1A, in which attractive intermolecular forces are effectively turned on with each input frequency. In the system illustrated in FIG. 1B, by contrast, each input frequency effectively “turns off” attractive intermolecular forces between the shuttle and the respective sites along the chain. For example, to move the shuttle from position 1 to position 2, frequencies ν₁ and ν₃ are applied simultaneously, thereby preventing binding at those positions. The shuttle tends to end up at position 2 over position 2′ because of diffusion, similar to FIG. 1A. The system in FIG. 1B is also bidirectional, and the shuttle can move between any two sites along the chain by a series of input signals having the correct pulse/frequency sequence.

FIG. 2 illustrates the working of a molecular shuttle device with a tethered shuttle. As shown in the figure, the shuttle is tethered between two subunits by covalent bonds or attractive intermolecular forces. Accordingly, three binding positions are possible, a (1 and 2), b (2 and 3), and c (3 and 1). For a tethered system, the shuttle can be designed such that it is not large enough to be coordinated by three subunits simultaneously and can only be tethered at one position at any given time. The loss of efficiency due to diffusion in solution is reduced or eliminated by the tethered approach.

In FIG. 2A, the input frequencies selectively “turn off” attractive intermolecular forces, for example, by cleaving covalent chemical bonds between the shuttle and the respective subunits along the chain. In the absence of an input, the binding affinity between the shuttle and any subunit is about the same. Therefore, applying an input signal of a specific frequency moves the shuttle away from the subunit to which it is tethered. For example, if the shuttle is at position a (i.e., between subunits 1 and 2), it is biased toward position 2 by breaking the bonding between the shuttle and subunit 1. The shuttle is still tethered to subunit 2, but can now move between subunits 1 and 3 by diffusion or a conformational space search. The longer the duration of input 1, the more the shuttle is biased towards being located at position b (i.e., between subunits 2 and 3).

As shown in the FIG. 2A, the shuttle can be moved from position b back to position a, or forward to position c, by inputting frequencies ν₁ or ν₃, respectively. The system is therefore bi-directional. Furthermore, the shuttle can be moved between any two positions along the chain by a series of input signals having the correct pulse/frequency sequence.

In FIG. 2B, the input frequencies selectively “turn on” attractive, non-covalent intermolecular forces between the subunits and the shuttle. In the absence of any inputs, the shuttle has no binding affinity toward any subunit. Such a system operates in a manner opposite that shown in FIG. 2A, in which attractive intermolecular forces are effectively turned off with each input frequency. For example, to move the shuttle from position a to position b, frequencies ν₂ and ν₃ are applied, thereby biasing binding toward position b. This system is also bi-directional as the shuttle can move between any two positions along the chain through application of the correct pulse/frequency sequence.

Numerous chemical species may be employed as subunits and shuttles in the molecular shuttle devices of the present invention. In some embodiments, the subunits include biomolecules capable of binding, preferably with specificity, to a complementary biomolecule on a shuttle. Complementary biomolecule pairs for use in the present invention are well-known in the art. Suitable types of complementary biomolecules include, but are not limited to, biomolecules independently selected from the group consisting of oligonucleotide sequences, including both DNA and RNA sequences, amino acid sequences, proteins, protein fragments, ligands, receptors, receptor fragments, antibodies, antibody fragments, antigens, antigen fragments, enzymes and enzyme fragments. Thus, the biomolecular interactions between the complementary biomolecule pairs include, but are not limited to, receptor-ligand interactions (including protein-ligand interactions), hybridization between complementary oligonucleotide sequences (e.g., DNA-DNA interactions or DNA-RNA interactions), and antibody-antigen interactions.

In some embodiments, the input signal used to alter the binding interactions between a biomolecule on a shuttle and a complementary biomolecule on a given subunit causes local heating of the biomolecule on the subunit to a temperature sufficient to overcome the non-covalent binding interactions between the complementary biomolecules. For example, the input signal may dehybridize bound oligonucleotides. In other embodiments, local heating of the subunit caused by the input signal induces the formation of non-covalent bonds between the biomolecule of a subunit and the biomolecule of the shuttle. In one such system, the solution may be maintained at a temperature low enough to disfavor binding between the subunits and the shuttle. For example, for a subunit/shuttle pair that bind through receptor-ligand interactions, the system may be maintained at a temperature that greatly slows down the binding kinetics. Similarly, for a subunit/shuttle pair that bind through enzymatic interactions, the system may be maintained at a temperature low enough to greatly reduce enzymatic activity. In systems such as these, a resolvable input signal may be used to locally heat the biomolecule of a given subunit to a temperature which favors binding to the biomolecule on the shuttle.

In certain embodiments, the molecular chains will be composed of identical connected biomolecules, each biomolecule having a molecular antenna attached thereto. Neighboring molecules have distinct molecular antennae, such that the input signal for neighboring subunits are resolvable. Suitable antennae for inducing local heating of biomolecular subunits include, nanoparticles, including semi-conductor quantum dots, nanowires, nanotubes, nanorods, fullerene or fullerene-like molecules, and the like. The nanoparticles may be metallic or magnetic nanopartices and may include as gold nanoparticles, silver nanoparticles, copper nanoparticles and cobalt nanoparticles. Other suitable antennae include, but are not limited to colloidal magnetite and supramolecular structures, such as manganese-oxide single-molecule magnets, polymers, and dye molecules, such as rhodamine. The dye molecules should be characterized by high quantum efficiencies with high photon counts.

The molecular antennae may be covalently attached to the biomolecules on their respective molecular subunits using conventional reaction schemes, many of which are well-known. For example, 1-ethyl-3-(3-dimethylaminopropyl)-carboiimide (EDC) or dicyclohexylcarbodiimide (DCC) coupling may be used between amines and carboxylic acids. Another coupling method is described in Nature, Vol. 415, pp. 152-155 (2002), which is incorporated herein by reference.

Suitable input signals for use with the nanoparticle antennae include, alternating radio frequency magnetic fields (AC-RFMF) and alternating radio frequency electric fields. By way of example, the mechanism through which an AC-RFMF input signal induces local heating of a DNA strand covalently linked to an antenna is described as follows. An AC-RFMF signal at 1 gigahertz (GHz) can trigger the coupling of gold nanoparticle antenna covalently linked to a DNA strand, thereby locally heating and dehybridizing DNA selectively over DNA without a covalently attached antenna (see, e.g., copending U.S. patent application Ser. No. 09/905,831, the entire disclosure of which is hereby incorporated by reference). Similarly, a magnetic nanoparticle antenna covalently linked to DNA may cause local heating and thereby trigger selective DNA dehybridization. This is similar to heating non-covalently linked magnetite to destroy biological molecules. In the presence of a magnetic field, the entire nanoparticle orients itself with the field along an anisotropic easy-axis in order to minimize its energy by having its magnetization along an easy-axis. If the nanoparticle is a multi-domain particle, heat can be generated by an AC-RFMF by hysteretic losses. Single-domain and superparamagnetic nanoparticles have different AC loss mechanisms. In the low frequency RF regime (˜sub-MHz), the nanoparticle causes local heating via Brownian losses as the magnetization tracks the field by rotation of the entire particle. In the high frequency RF regime (˜sub-GHz), heat can be generated by Neél losses in which the magnetization tracks the field by rotating within the nanoparticle. In the microwave regime, heat can be generated by ferromagnetic resonance (FMR) coupling between the nanoparticle and the field. These relaxation modes have resonance frequencies associated with them, at which maximum power dissipation occurs. These resonance frequencies depend on the size, shape, crystalline structure, and chemical composition of the nanoparticle, the magnetic field strength (both parallel and longitudinal components), and the viscosity of the solvent. If there is sufficient energy coupled into the system at resonance, but not at off-resonant frequencies, magnetic nanoparticles may provide RF antennae having response frequencies resolvable from the response frequencies of gold antennae. Likewise, a second magnetic nanoparticle with different resonance frequencies may provide a third input. A three-antenna system thus can employ, for example, gold nanoparticles, colloidal magnetite, and manganese- oxide single-molecule magnets as antennae.

Remote electronic control over the hybridization behaviour of DNA molecules, by inductive coupling of a radio-frequency magnetic field to a metal nanocrystal covalently linked to DNA is described in Nature, Vol. 415, pp. 152-155 (2002), the entire disclosure of which is incorporated herein by reference.

The biological applications of a covalently attached gold nanoparticle to DNA can be replicated using magnetic nanoparticles and single-molecule magnets as antenna. Dodecanuclear manganese single-molecule magnets, such as Mn12-ac (i.e., Mn₁₂O₁₂(CH₃O₂)₁₆(H₂O)₄), make especially good antennae because of their exact and small size. The use of magnetic nanoparticles is desirable in biological applications (versus diamagnetic metals) because the ability to generate heat at frequencies less than one GHz lessens global heating or any other microwave effect that can be harmful to cells.

Dye molecules may also serve as optical antennae. Local heating may be accomplished by optically exciting dye molecules covalently attached to DNA to selectively dehybridize the DNA. Preferred dyes are organic molecules and polymers with narrow or tunable absorption spectra.

In some embodiments, the subunits and the shuttle experience biomolecular binding interactions that are selectively affected by chemical input signals. In these embodiments, the subunits are chemically distinct and are capable of interacting directly with resolvable chemical input signals without the need for covalently bound antennae. For example, a shuttle may be capable of binding to different biomolecules on different molecular subunits under distinct pH conditions. The binding in such systems may occur, for example, through receptor-ligand interactions or antibody-antigen interactions. The inputs for these systems may consist of chemical species, such as acids, bases and neutralizers, that are capable of adjusting the pH of the system.

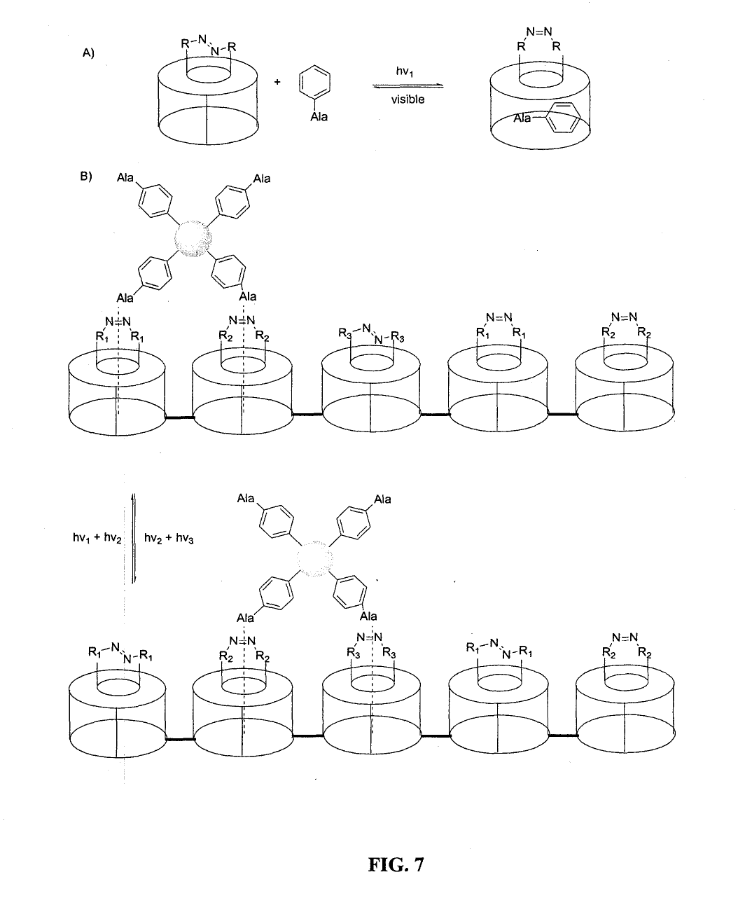

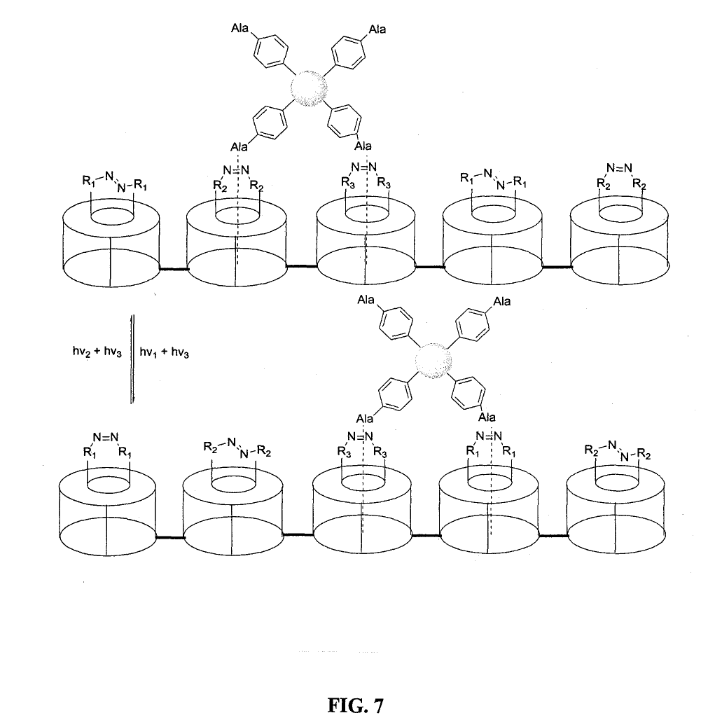

In some embodiments, the subunits and shuttle undergo small molecule binding interactions rather than biomolecular interactions. As one of skill in the art will recognize, a great variety of chemical species may be used to produce the subunit/shuttle binding pairs in such systems. By way of illustration, examples of a three such systems are discussed in conjunction with FIGS. 7-9 below.

The molecular shuttle devices may be used as switches and molecular assembly lines. A switch may be made from a molecular chain having two distinct binding positions for a shuttle, one corresponding to the “on” position and one corresponding to the “off” position. Thus, a switch may be formed from a chain of two or three molecular subunits, depending on whether the shuttle binds individually to each subunit in the chain, or between adjacent pairs of subunits. The position of the shuttle represents the state of the switch. Any system with two resolvable inputs can function as a bi-stable switch.

A molecular assembly line refers to a molecular system that is capable of moving a shuttle step-wise along a chain of subunits. The shuttle in a molecular assembly line may serve as an attachment point for a growing molecule. Thus, the shuttle may be any physical object, such as a bead, capable of binding to a growing molecule and capable of moving to any position along a molecular chain in response to an appropriate input signal or series of input signal. As the shuttle moves to different binding positions, the growing chain is brought into contact with or into close proximity to molecular building blocks which may be added to the molecule. In some instances, chemical inputs, such as catalysts, including biocatalysts, may be introduced into the system once the growing molecule and a building block have been brought together by the molecular assembly line, in order to facilitate the reaction of the growing molecule with the the building block. Examples of molecules that may be assembled using a molecular assembly lines include polymers, where the molecular building blocks are monomers, and oligonucleotides, where the molecular building blocks are nucleotides.

A molecular assembly line may be made from a chain of molecular subunits that define at least three binding positions. Thus, a molecular assembly line may be made from a chain of three or four subunits, depending on whether the shuttle binds to the subunits individually or between adjacent subunit pairs. One embodiment of a molecular assembly line includes a repeating pattern of at least three subunits linked together through a connecting group, such as a polymer chain (i.e., a polymeric backbone). Bi-directional molecular assembly lines can be constructed. Any untethered system with three resolvable inputs can function as a molecular assembly line because it is bi-directional and, using proper input sequences, the shuttle may reach any position in the system when the current position is known. The position of the shuttle can be determined by spectroscopic or microscopic means. Since the shuttle can be bound to any position along the molecular assembly line, the shuttle can also serve as a site for a molecular assembly process. For example, the shuttle may be bound to a molecular building block, such as a bead that serves as a solid-phase support for oligonucleotide synthesis. If the polymeric chain is a closed loop, a plasmid-like device can be constructed.

A molecular assembly line composed of repeating sections of three subunits, wherein the input signal for each subunit is resolvable from the input signals for the other two subunits is particularly desirable because it provides an assembly line that allows for bi-directional movement of a shuttle without the need for more than three distinct input signals.

FIG. 1A shows a bi-directional molecular assembly line using three inputs. The molecular assembly line is a polymer of three repeating subunits: -(1-2-3)ₙ-. Here, the position of each subunit (i.e., 1, 2, 3, 1′, 2′, 3′) refers to the position at which the shuttle can interact and reside.

A tethered system with three resolvable inputs is bi-directional and can reach any site in the system if the current position is known. Thus, such a tethered system can function as a switch or as a molecular assembly line. Untethered systems of a molecular assembly line using the same components are diffusion limited, whereas tethered schemes are not. FIG. 2 schematically illustrates a tethered molecular switch and a bi-directional molecular assembly line with three inputs. The bi-directional molecular assembly line includes a polymer of three repeating subunits: -(1-2-3)ₙ-.

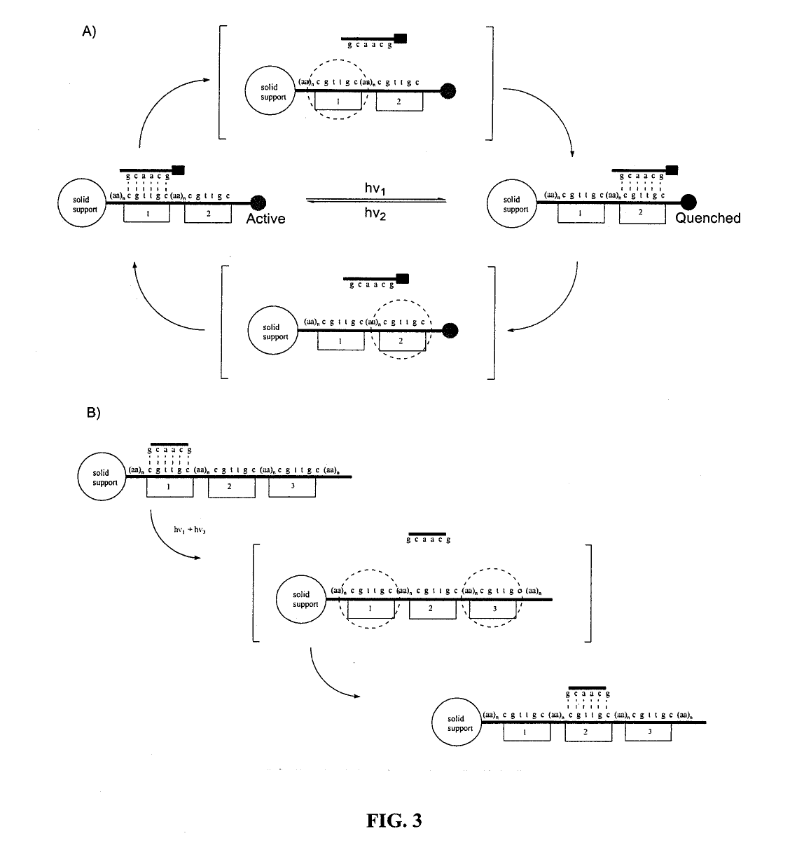

Specific embodiments of molecular switches, assembly lines, and memory devices in accordance with the present invention will now be presented with reference to FIGS. 3 through 11. These embodiments are provided for illustrative purposes only and are not intended to limit the scope of the invention.

As illustrated in FIG. 3, the present invention features techniques for constructing bi-stable switches and molecular memory, both with simultaneous and in-situ read-out mechanisms. FIG. 3 shows an untethered DNA assembly line using a DNA strand (GCAAG) as the shuttle and a bi-stable molecular switch using optical and RF inputs, with optical readout. Each subunit includes the complementary strand (CGTTCG) of the shuttle and an RF or optical antenna. Poly-adenine spacing groups (AA)ₙ are placed between each subunit. The subunits may be connected by polymerized them into the repeating pattern by sequential enzymatic ligation, or by sequential bioorganic addition employing capping-group chemistry.

FIG. 3A schematically illustrates a bi-stable DNA switch with an optical readout. To synthesize the switch, subunit 1 (i.e., DNA fragment+antenna 1+spacer) is attached to a solid phase support. Next, the shuttle, a complementary strand with a quencher attached, is hybridized to subunit 1. Subsequently, subunit 2 (i.e., DNA fragment+antenna 2+spacer+fluorophore) is attached to subunit 1. Switching is accomplished as shown in FIG. 1B. If the shuttle is in position 1 and ν₁ is input, the shuttle will move to position 2. There is a high binding affinity between the shuttle and subunit 2 due to hydrogen-bond formation or hybridization in the absence of local heating. The input 1 may be removed once the shuttle has reached position 2, and vice-versa. The switch is therefore bi-stable. If the fluorophore is active, the switch is in position 1. A quenched fluorophore indicates that the shuttle is in position 2.

FIG. 3B shows the mechanism of how an untethered, bi-directional molecular assembly line can be constructed according to FIG. 1B. In order for the switch to function properly, the length of the shuttle should be shorter than the thermal radius (represented by the dotted circles in FIG. 3) of the antenna. The length of the spacer should be long enough such that the thermal radii of the antennae of two adjacent subunits do not overlap. Furthermore, the spacer should be long enough such that the fluorophore is not quenched when the shuttle is in position 1.

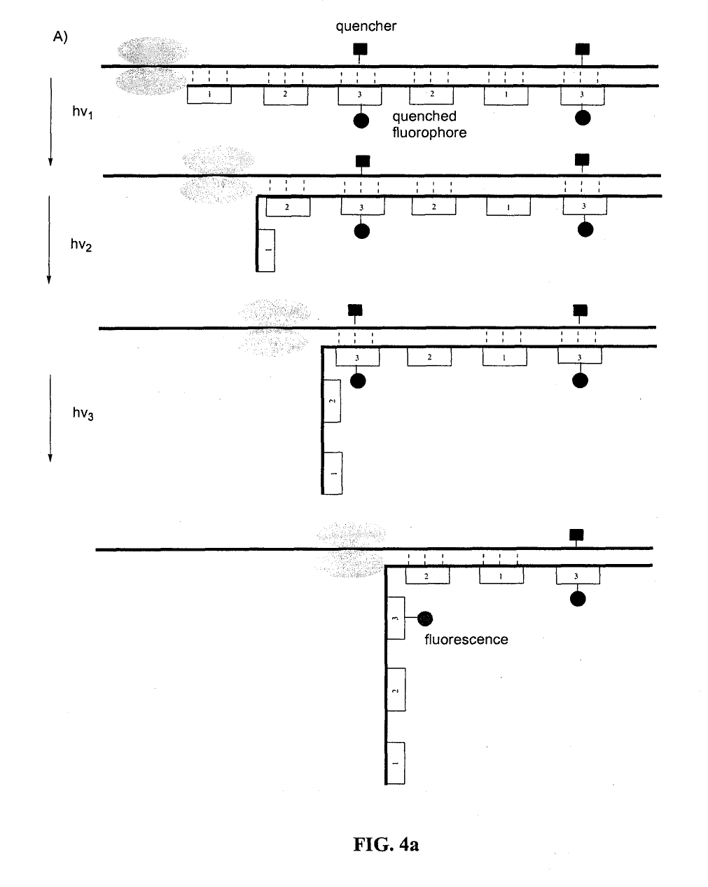

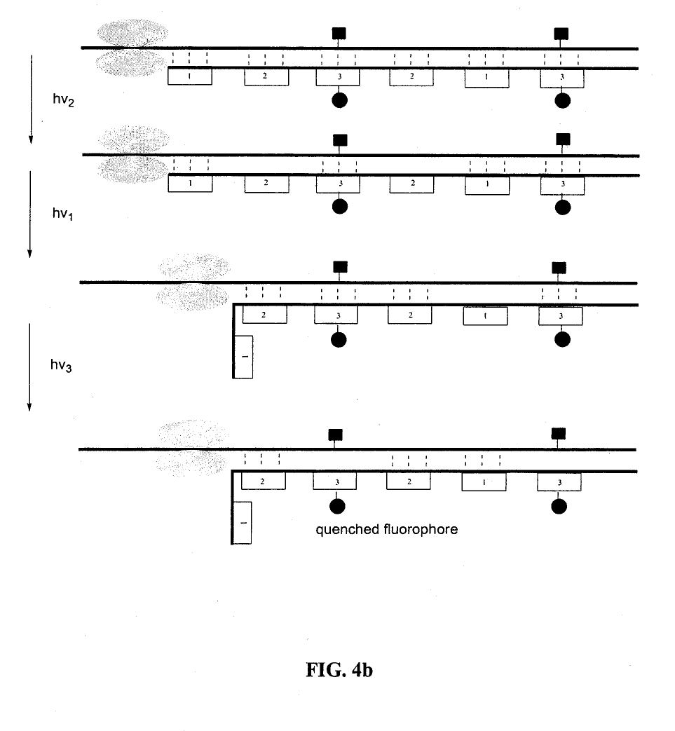

FIG. 4 schematically illustrates how molecular memory can be constructed using DNA. In the illustrated system, an DNA strand of three subunits represents one bit of information, in which 1-2-3 is defined as “zero” and 2-1-3 is defined as “one.” Therefore, an DNA strand comprised of N subunits can contain 2^{N/3} bits of information.

FIG. 4A schematically illustrates how the memory element is read. Once the DNA strand (DNAₘₑₘ) is “written,” it is hybridized to its complementary DNA strand (DNA′) with no antennae linked thereto. The memory is read by a biological enzyme, which typically moves along DNA′ with a forward bias. If the enzyme is in its starting position (see top of FIG. 4A), it is sterically prevented from moving along DNA′ because DNA′ and DNAₘₑₘ are hybridized. If ν₁ is applied, DNAₘₑₘ and DNA′ will dehybridize at position 1, and the enzyme moves along DNA′ until it reaches position 2. The enzyme cannot move forward due to steric hindrance until ν₂ is applied. The DNA site is blocked, much like in anti-sense therapeutics. Since the enzyme has a forward bias, a “1” sequence cannot be differentiated from 1-1, nor can “2” be differentiated from a 2-2 sequence. Subunit 3 therefore acts as a “domain wall.”

If a fluorophore is attached to every subunit 3 of DNAₘₑₘ and a quencher is linked to every corresponding position along DNA′, the current bit being read can be determined by the intensity of the fluorophore emission. For example, if no bits have been read, every fluorophore is quenched. After the first bit is read, one fluorophore per memory element will fluoresce. After the second bit is read, the emission intensity will double, and so forth. As is the case in FIG. 3, the memory is stable.

FIG. 4B shows how a “zero” bit can be differentiated from a “one” bit. If the first bit of the memory element is a “zero” (1-2), but the pulse sequence is for a “one” (2-1), the enzyme will not be at its proper position at the end of the “one” pulse sequence. Initially, ν₂ is first applied. The first subunit along DNAₘₑₘ is still hybridized to DNA′, so the enzyme is still at its initial position. Next, ν₁ is applied, and the enzyme moves one subunit along DNA′. Applying frequency ν₃ will not move the enzyme, as can be seen in FIG. 4B (bottom). Therefore, the first bit “zero” (1-2) is not properly read after the successive pulse sequence “one” (ν₂, ν₁), which can be determined because there is no fluorophore emission detected.

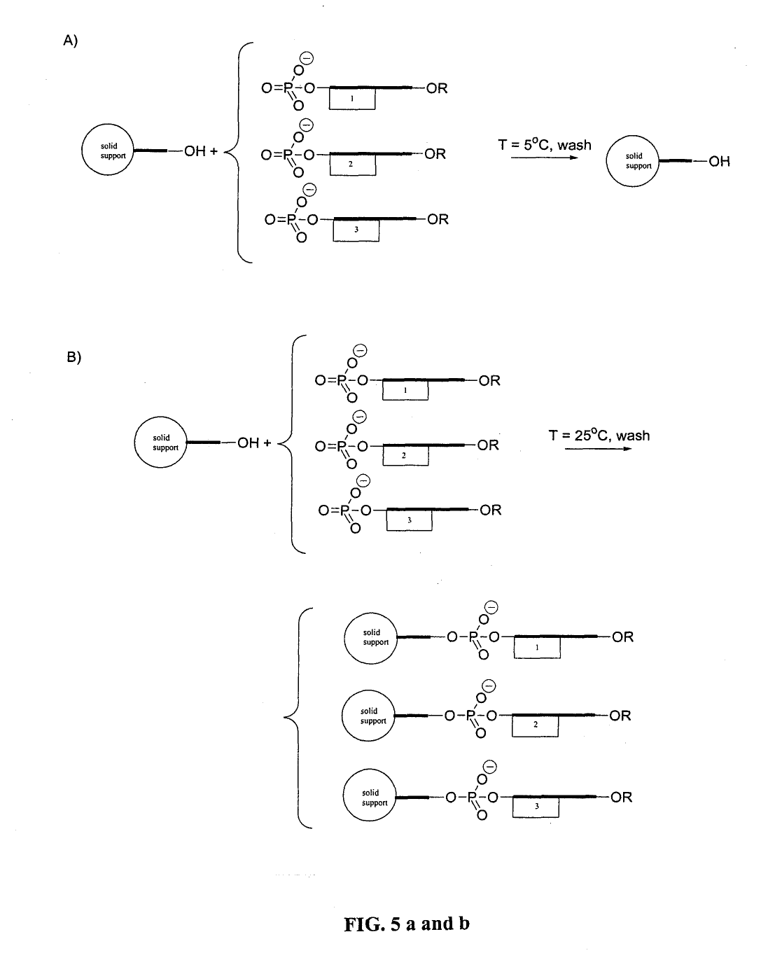

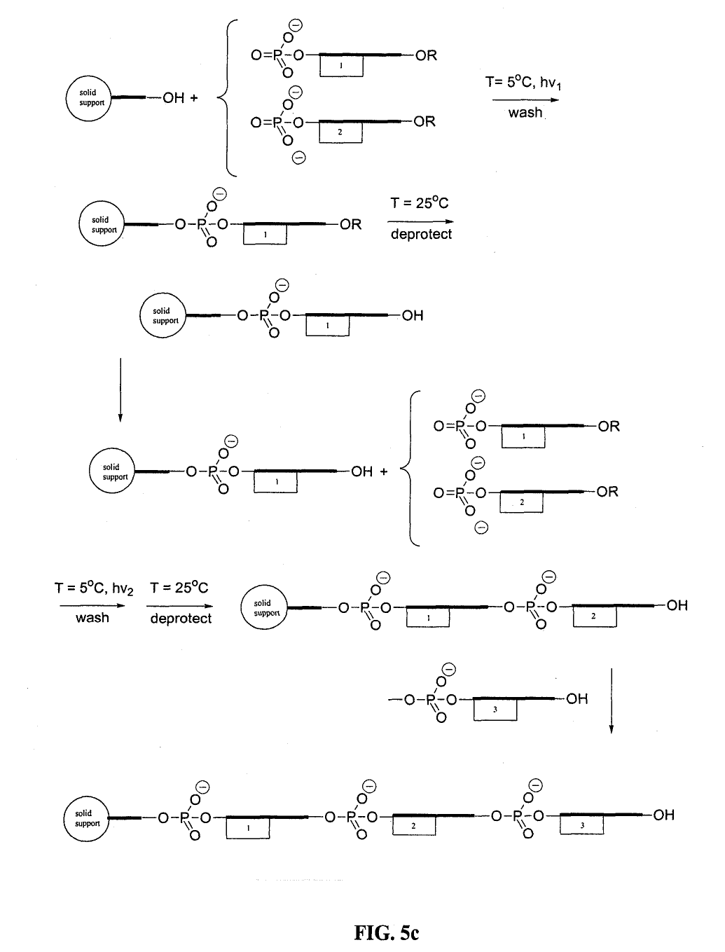

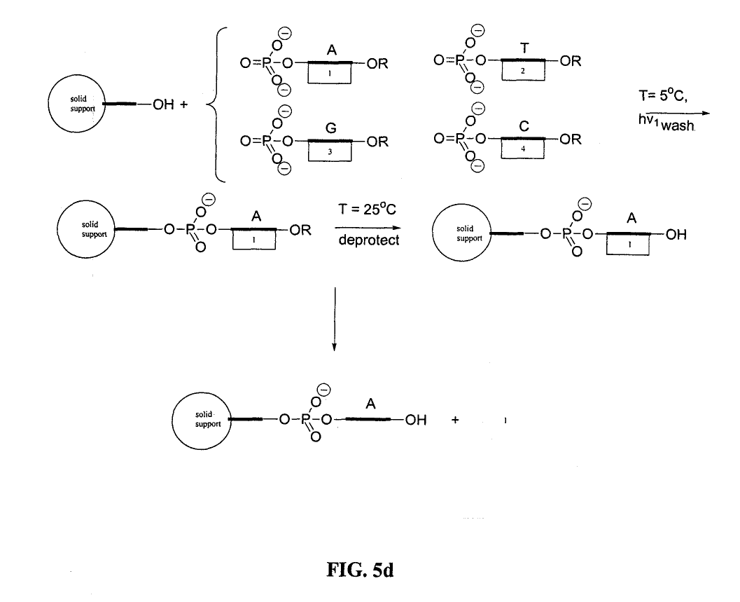

FIG. 5 schematically illustrates molecular memory constructed of DNA strands, which can be “written” by selective incorporation of DNA strands by polymerase or by bioorganic synthesis (e.g., solid-phase phosphoramidite chemistry). This is accomplished by kinetically biasing the addition of subunit 1 or 2 to DNAₘₑₘ during solid phase synthesis. A “zero” pulse sequence (ν₁, ν₂) can write a “zero” bit.

In FIG. 5A, the solid support is in solution with the same molar concentration of subunit 1 and subunit 2, both with protecting groups at their 3′ ends. The protecting groups prevent polymerization of individual subunits to anything but the growing chain. The solution is kept at a temperature low enough to greatly slow down the kinetics of the addition, or in the case of enzymatic synthesis, kept below the ideal temperature for enzymatic activity. In the absence of any local or global heat, there will be very little strand addition after the support is washed. In FIG. 5B, because the solution is kept at room temperature, there is an equal probability of strand addition to the growing chain.

In FIG. 5C, the first bit of DNAₘₑₘ is “written” as a “zero” by kinetically biasing the first subunit that attaches to the solid support to be a subunit 1. The figure is drawn for a 3′-OH addition to a 5′-phosphate (e.g., enzymatic addition or 5′-phosphoramdite). If ν₁ is applied, there is a local thermal gradient created around the 5′ phosphate of subunit 1. This local temperature increase about the phosphate of subunit 1 kinetically favors its reaction with the solid support over the phosphate of subunit 2. In an enzymatic system, the temperature approaches the optimal temperature of activity. In a bioorganic system, a temperature increase by 10° C. increases the reaction rate by a factor of two. Therefore, a local temperature of 40° C. will favor this selective addition by a factor of 16. Selective addition can be determined by spectroscopic or microscopic means. Selective addition can demonstrate that molecular memory has been “written,” because the absence of any input does not favor any DNAₘₑₘ sequence.

Following the washing and deprotecting of DNAₘₑₘ, which is currently “solid support+subunit 1,” DNAₘₑₘ is deprotected. The steps are repeated as described above, except with frequency ν₂. Finally, subunit 3 is ligated at room temperature to finally “write” the first bit as a “zero.” The absence of subunit 3 from the previous steps does change the fact that molecular memory has been written because selective ligation of subunits 1 or 2 over subunit 3 is irrelevant. Subunit 3 is merely functioning as an analog to a domain wall.

FIG. 5D illustrates the first nucleotide base addition in a “one-pot” oligonucleotide synthesizer. Four antennae are needed, one for each nucleotide. Each antenna, one through four, is capped by a nucleotide: adenine, guanine, cytosine, and thymine. Adenine is the first nucleotide added to the solid support by applying ν₁. In order to free the desired oligonucleotide from the attached antennae, a removal step is introduced into the process described in FIG. 5C. Suitable cleaving agents (depending on the linkage between nanoparticle and oligonucleotide) break the bonds between antennae and oligonucleotide without damaging the oligonucleotide.

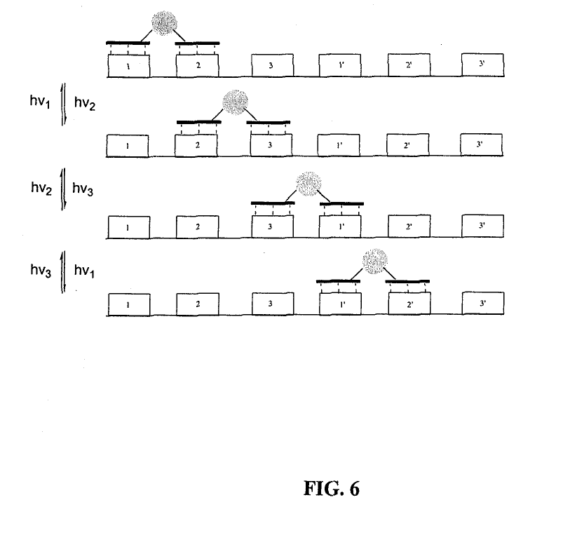

FIG. 6 schematically illustrates a tethered molecular assembly using the DNA polymer as described in FIG. 3B as the assembly line and a DNA-capped nanoparticle as the shuttle. The capping group is the complementary strand to the assembly line subunits. The system operates under the same principles as in FIG. 2A, where the intermolecular forces being broken or “turned off” are hydrogen bonds in DNA hybridization. As is the case in FIG. 3, the positions are stable. Any solid can serve as a shuttle so long as it can be covalently attached to moieties that interact with the molecular assembly line.

FIGS. 7A and 7B schematically illustrate a tethered molecular assembly line using a cyclodextrin polymer and optical inputs. Each subunit includes a cyclodextrin monomer with an azo compound serving as a “gate” to its cavity. The gate is open when the azo compound is in its trans-, or Z-isomer conformation. The gate is closed when the azo compound is in its cis-, or E-isomer conformation. The shuttle in FIG. 7 is a nanoparticle capped with phenylalanine (FIG. 7A). Three azo compounds that undergo cis-trans isomerizations at three resolvable frequencies are used as subunits in the molecular chain. The gate need not be an azo compound. Molecular recognition or guest-host interaction between cyclodextrin and phenylalanine are favored when the azo compound is in its Z-isomer. The system shown in FIG. 2B involves the intermolecular force in molecular recognition. The intermolecular force is “turned on” by opening the gate of the azo compound with ultraviolet light, and “turned off” by closing the gate using visible light as in input signal. There are several azo-benzene derivatives with resolvable UV absorption bands, such as the “Disperse Orange” and “Disperse Red” class of azo dyes. Additional selectivity between azo compounds may be achieved by employing two-photon absorption (TPA), since the two-photon cross sections of such compounds are known to depend on the R-groups. Optical-shape pulsed femtosecond lasers may also be employed as input signals to improve wavelength selectivity conferred by altering the R-groups on the azo dyes. Discussions of Azo compounds and cis-trans-isomerization of azo compounds in response to photoirradiation are provided by De Boni, et al. in Chem. Phys. Lett., 361, 209-213 (2002) and by Asanuma et al., in Tetrahedron Letters, 40, 7995-7998 (1999), the entire disclosures of which are incorporated herein by reference.

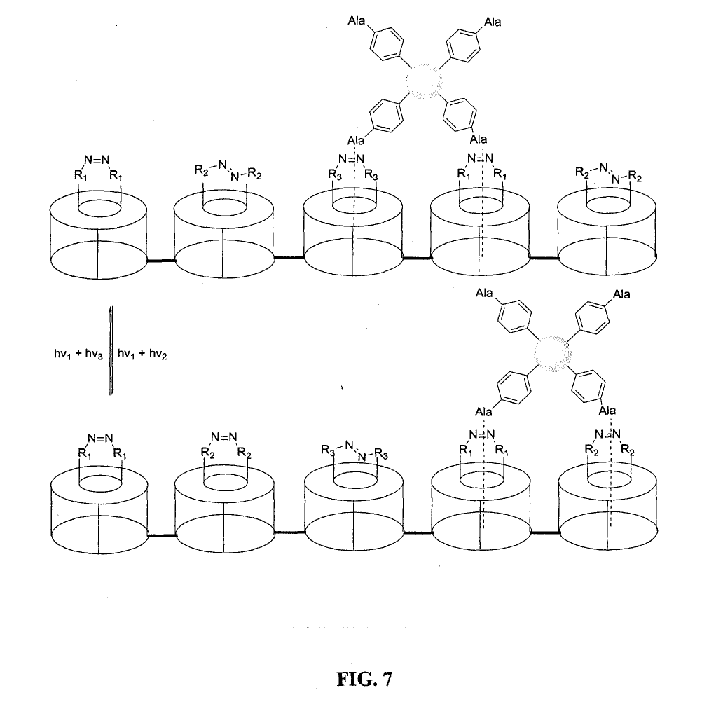

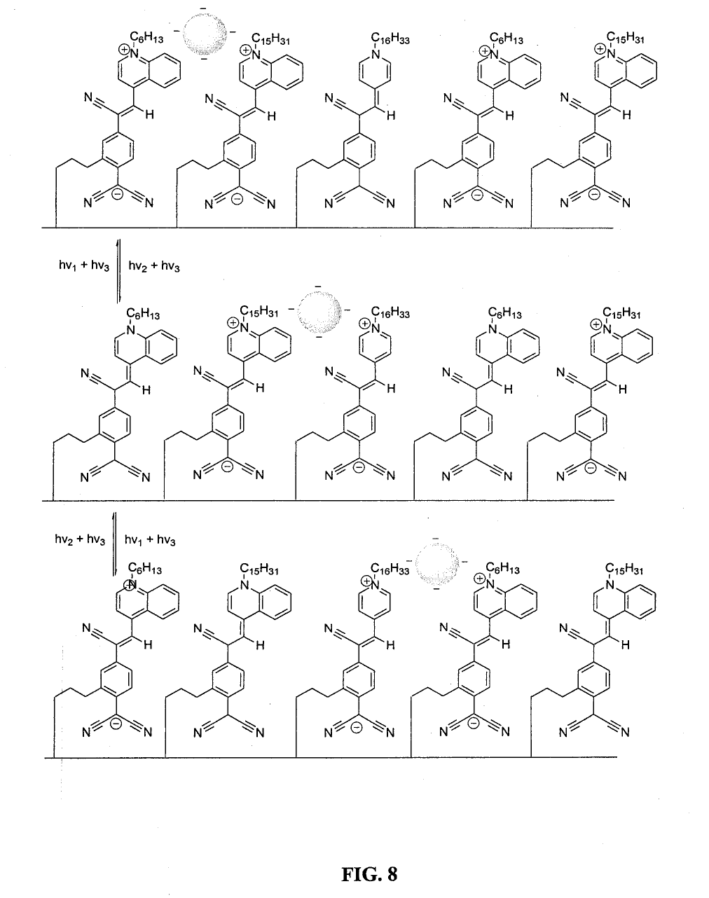

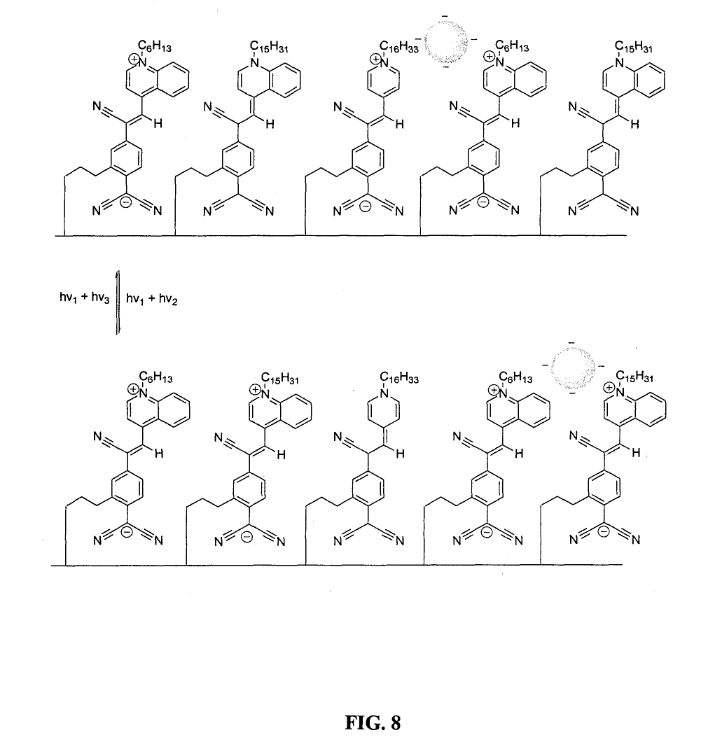

FIG. 8 shows a tethered molecular assembly line using photochromic zwitterions as the subunits of the assembly line and a negatively charged nanoparticle as the shuttle. The structure shown in FIG. 8 can function as an electrostatic motor using optical inputs. Photochromic zwitterions can be readily synthesized from commercially available materials. Upon input of ultra-violet light, zwitterions are created. Visible light returns the molecules to their non-zwitterionic forms. Photochromic zwitterions absorb in a large and tunable range of frequencies and are highly resolvable because their ultraviolet absorption peaks have narrow full-width-half-maximums. The molecular assembly is synthesized such that the positive charge created by the optical input is oriented away from the polymer backbone. This system follows the principles of FIG. 2B, in which the intermolecular force is electrostatic attraction between the negatively charged shuttle and the positive charge of the zwitterions. Because the motive force is electrostatic attraction, this molecular assembly line is also considered a molecular, electrostatic motor.

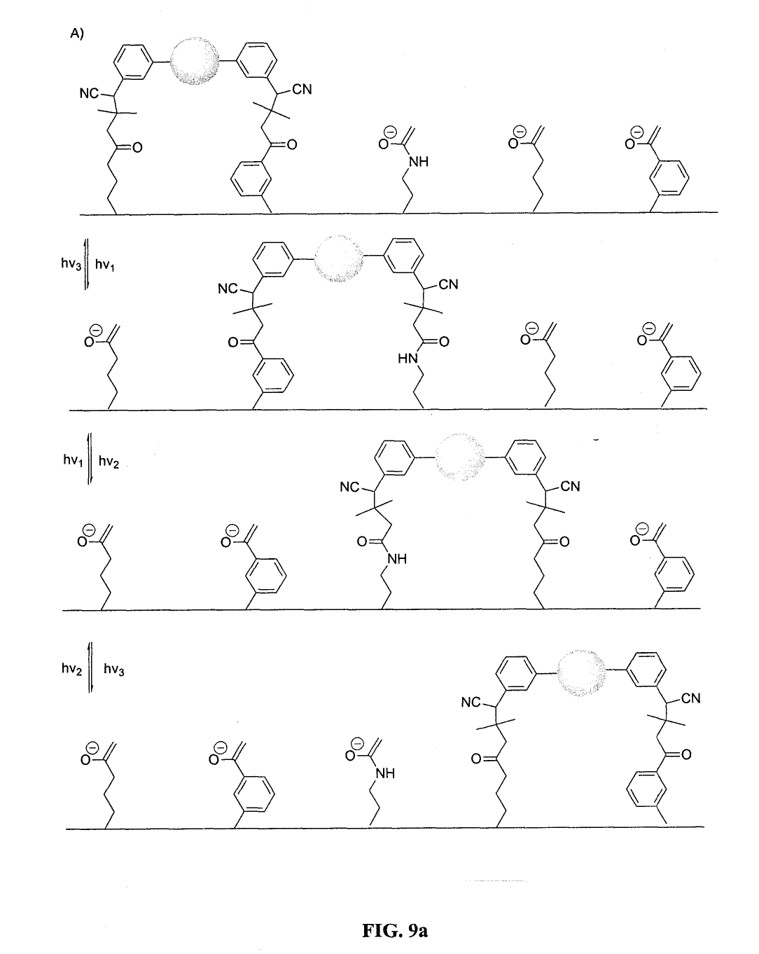

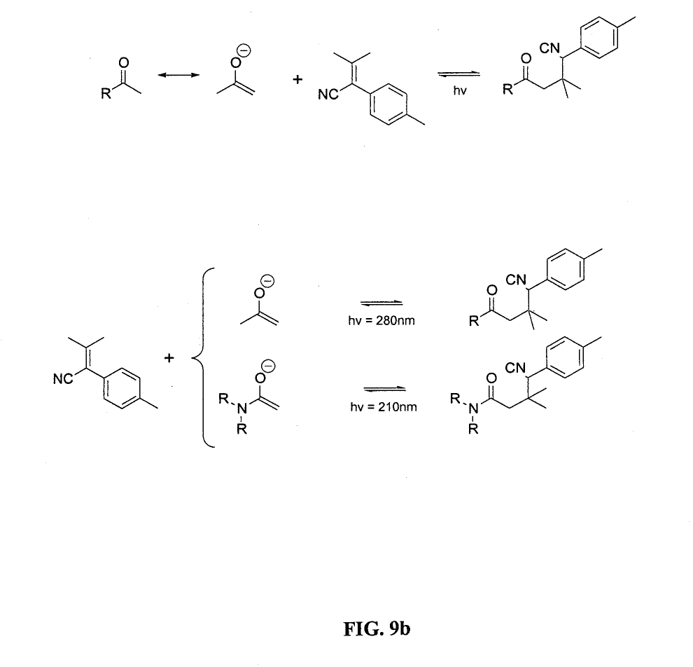

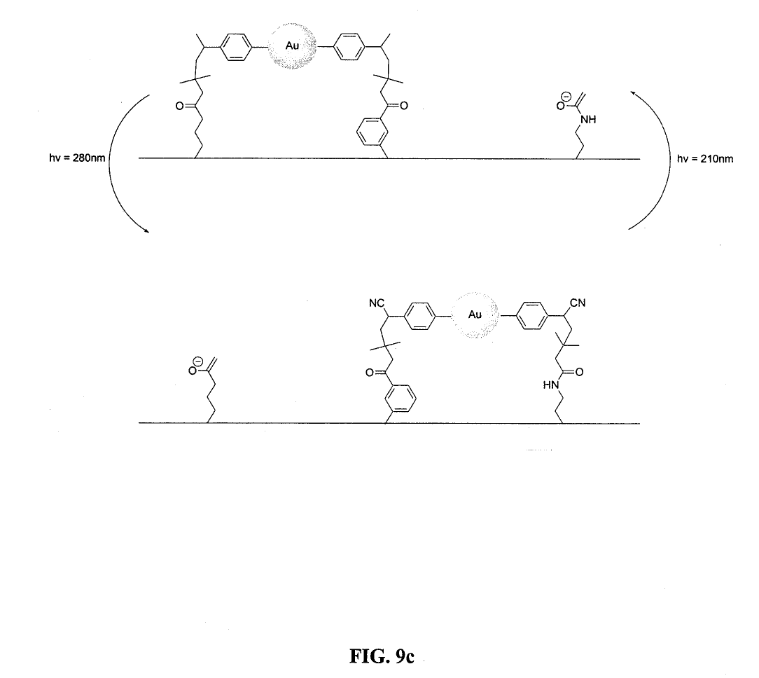

FIG. 9A shows a covalently tethered molecular assembly line including subunits with chemical moieties that can form photo-cleavable bonds with the alkene capping groups of the nanoparticle shuttle. FIG. 9 also shows a bi-stable molecular switch with spectroscopic readouts. This system follows the principles of FIG. 2A, in which the shuttle is tethered by covalent bonds. Because the shuttle is always tethered to the assembly line by at least one covalent bond, diffusion in solution can be eliminated. One bond is “turned off” by photo-cleavage (e.g., the covalent bond between subunit 1 and the shuttle). The newly formed bonds are covalent and are stable in the absence of any input.

FIG. 9B and Example 2 below illustrate the chemistry governing the system illustrated in FIG. 9A. A carbonyl group is transformed into its enolate or amidine form in basic buffer conditions, which can then react with an alkene by a Michael addition to form an acylated product. The carbonyl group can be photo-cleaved to yield the original two products at the maximum ultraviolet absorption wavelength (or frequency) to yield the original alkene and carbonyl. The absorption wavelength can be tuned by altering the R-group attached to the carbonyl groups of the molecular assembly line subunits. The rates of the photo-cleaving, and Michael addition between the subunits and the alkene capping groups of the shuttle, can be selected not to differ significantly between subunits. The general molecule in FIG. 9B has been designed as it is for various reasons. The cyano (or nitrile, —CN) group at the 3-carbon of the Michael acceptor promotes the addition to the 2-carbon of the alkene. The aromatic group at the 5-carbon, and the methyl groups at the 4-carbon of the Michael product promote the Norrish Type II photo-cleavage pathway (as opposed to Norrish Type I cyclization).

Wavelength selective photo-cleavage of covalent bonds can be used to selectively control product formation. First, desired carbonyl compounds are made from an equimolar mixture of an alkene and an enolate and amidine. For example, one equivalent of an alkene is placed in a solution with an enolate and an amidine. The resulting carbonyls are roughly equal in concentration in the absence of any input. Irradiating the solution with 280 nm light cleaves the ketone compound selectively over the amide. The resultant alkene can react with the enolate or the amidine. Over time, however, the alkene is biased to react with the amidine. After irradiating with the 280 nm light for an excessive time, the reaction is quenched. Spectroscopic or chromatographic means can be used to determine whether selective bond cleavage and biased bond formation have been achieved.

In FIG. 9C a polymer containing subunits 1, 2, and 3 is used as a bi-stable molecular switch. The position of the alkene-capped nanoparticle is switched between positions a and b. The position of the switch can be determined by spectroscopy.

Because the bonds are expected to cleave between 200-300 nm, a two-photon approach uses 400-600 nm radiation from an ultraviolet laser, which is more versatile than the much more expensive 200-300 nm tunable ultraviolet laser (lower laser cost, higher laser stability, less destructive). Shaped-pulsing of lasers photo-cleaves covalent chemical bonds more effectively than non-shaped pulsing.

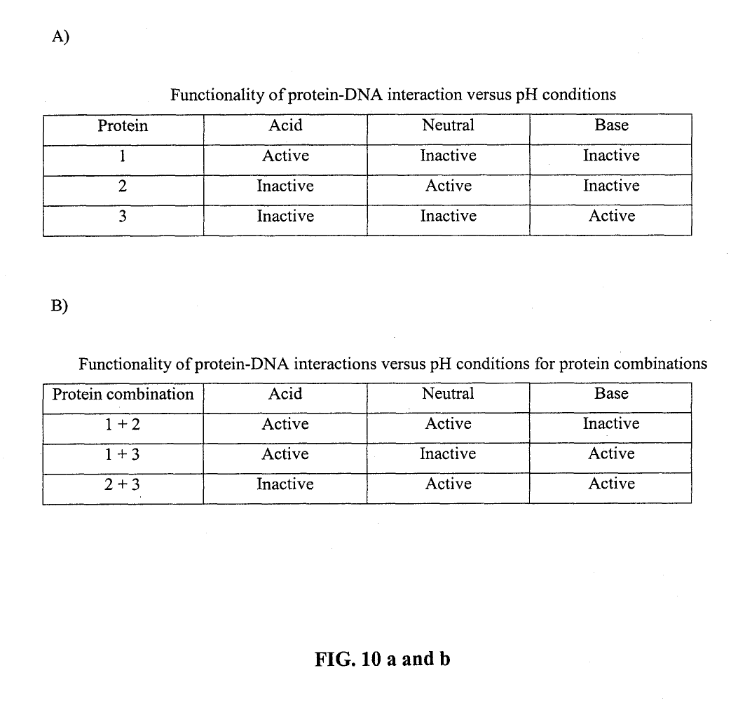

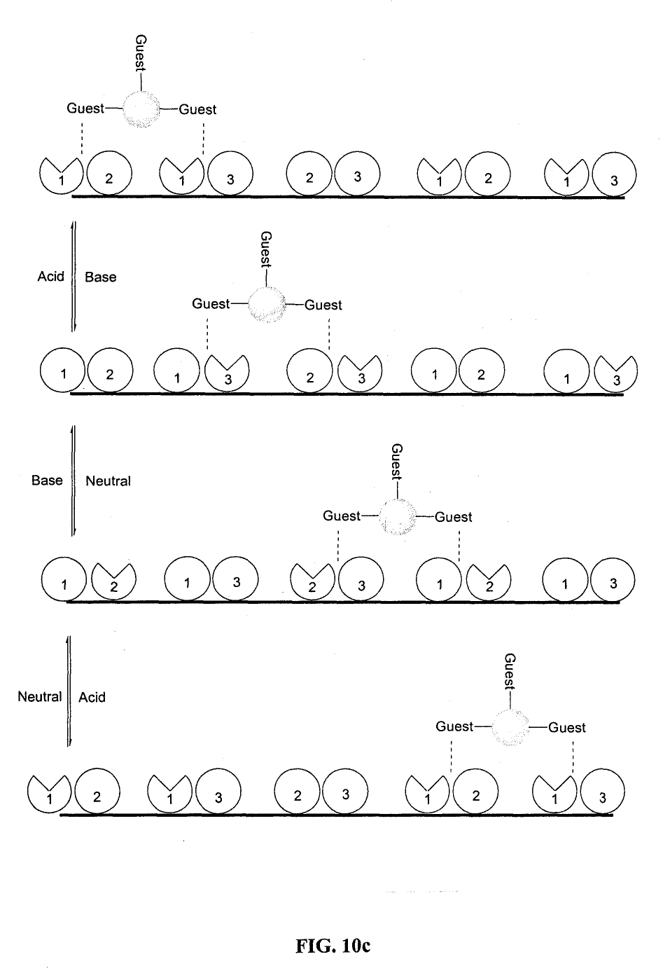

FIG. 10 shows a tethered molecular assembly line with chemical inputs. The assembly line contains proteins whose conformations are pH-dependent, allowing proper protein-oligonucleotide interactions at only one pH range (acid, neutral, basic), as shown in FIG. 10A. While most proteins function at only neutral pH levels, proteins that function properly only under acidic or basic conditions can be found, for example, in certain bacteria known as extremophiles. Each subunit along the line includes a combination of two different proteins. Each combination is completely inactive at one pH range, as summarized in FIG. 10B.

Using such an approach, FIG. 10C shows how a shuttle can move along a tethered molecular assembly line in the manner shown in FIG. 2A, using pH-buffer ranges as chemical inputs. This approach uses biological proteins in coordinating the movement of a shuttle, but the utility of the approach as a molecular assembly line is limited because it is not a remote system and because its pH range of operation is very broad.

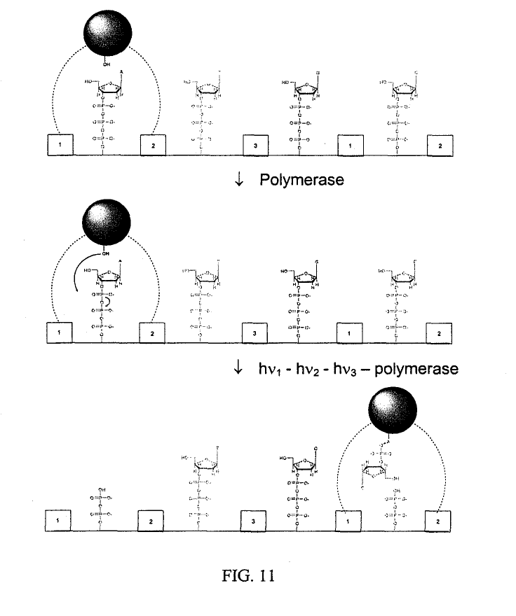

FIG. 11 shows an example of how the shuttle of a molecular assembly line can be used as a solid-phase synthesis support for chemical synthesis that is not limited by diffusion. The shuttle could be a controlled-pore glass sphere with a growing DNA strand and the four nucleic acids could be located at reactive sites along a molecular chain; nucleic acid of choice may be added to the growing chain in a selected order by moving the bead to a desired binding position along the molecular chain and then introducing polymerase into the system. In the figure: 1) Top: the support is positioned over an adenine residue; Middle: Polymerase is added and the nucleotide is now on the solid-support while a diphosphate is left on the polymer backbone; and Bottom: The support is positioned over a cytosine residue and the coupling step occurs again.

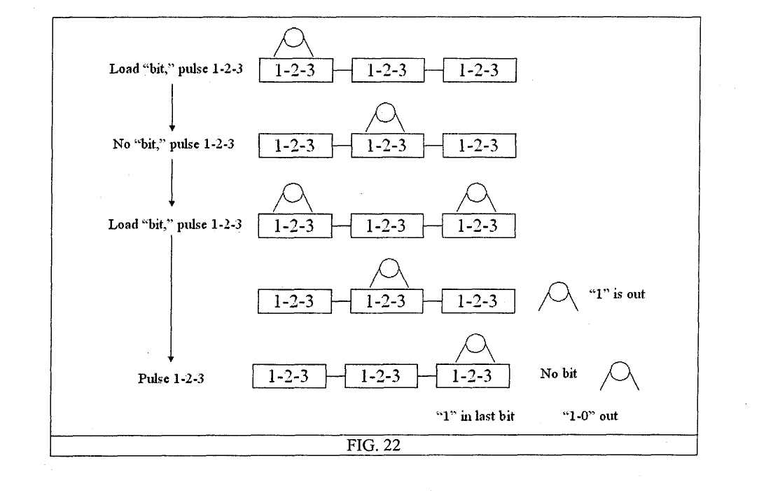

FIG. 22 shows how the shuttle devices provided herein can be used to build non-volatile, molecular shift register memories. A three-bit shift register memory is shown in the figure, in which a bead, as a physical object, represents a “1,” and the absence of a bead represents a “0.” A bead is loaded onto the chain, and then a 1-2-3 input is pulsed, representing the writing and shifting of a “1.” No bead is loaded, and then a 1-2-3 input is again pulsed, representing writing of a “0” and shifting of both bits to the right. Note that both bits are shifted by the same input, making the device a shift register. Another bead is loaded, and after the 1-2-3 pulse, the initial bead is cleaved completely from the chain as the other two bits are shifted to the right along the chain, representing a “1” bit out. No bead is cleaved from the next 1-2-3 input, representing a “0” bit. After this pulse, the final “1” is still located (stored) along the three-bit shift register chain.

Another aspect of the invention provides a molecule having the structure:

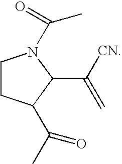

The molecule is capable of acting as a toggle switch according to the following scheme:

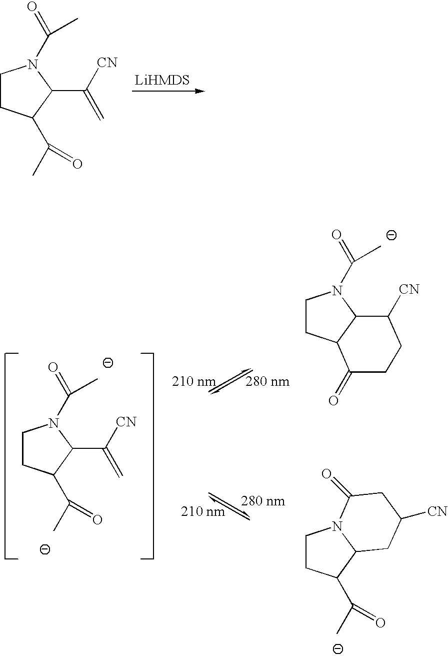

The toggle switch (or flip-flop switch) is driven by UV-irradiation as follows. Such a switch could be used for optically written memory. When the ketone and amide functional groups are in their enolate and amidine forms (e.g., in the presence of LiHMDS base), both can act as Michael donors to the Michael acceptor alkene, resulting in two potential cyclized Michael products. The relative product equilibrium can be driven by the wavelength of UV-irradiation. It is possible to trap the radical intermediate of the reaction by simultaneously irradiating the toggle switch with both wavelengths. The methyl groups that provide steric hindrance (γ-carbons with respect to carbonyls) and the aromatic group (6-carbon) that promotes hydrogen abstraction adjacent to the nitrile are not required. 3D-modeling (ChemDraw 3D Ultra, Cache) showed that products of the photoreaction following a Norrish Type I path, or a path via 1-5, 1-6, or 1-7 bi-radical intermediates, are unfavorable because of great ring strain.

A method for making the molecule according to the following synthetic scheme is also provided:

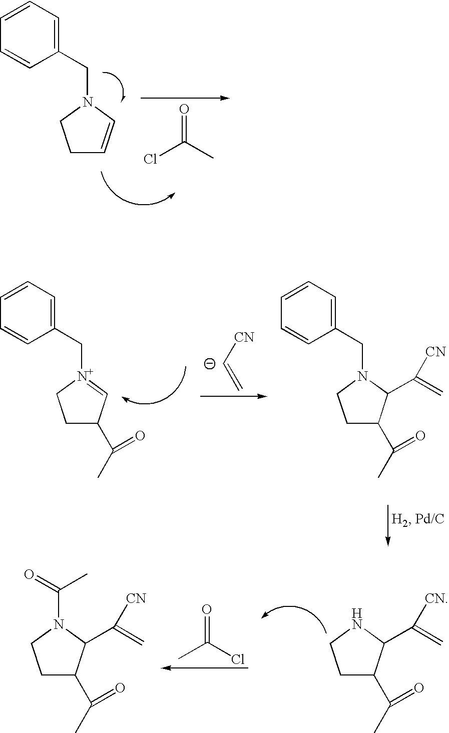

### EXAMPLES

### Example 1

### Selective Dehybridization of Nucleic Acids by Resolvable RFMF Signals

This example demonstrates the selective dehybridization of nucleic acids for a nucleic acid subunit having a cobalt nanoparticle antenna covalently attached thereto.

Cobalt Nanoparticle Synthesis:

All reagents were of the highest purity available and were used as received from Sigma-Aldrich unless otherwise indicated. Reactions under nitrogen were performed in a MBraun UniLab glove box. High-resolution TEM micrographs were attained using a JEOL 2010 TEM on copper/Formvar grids from Ladd Research Incorporated. FTIR data was taken using a Digi-Lab Excalibur FTS 3000 (KBr, 64 scans, 4 cm⁻¹ resolution).

Synthesis of cobalt nanoparticles was performed under nitrogen. Cobalt chloride (COCl₂, 1.0 mmol) and 11-aminoundecanoic acid (2.0 mmol) were added in octyl ether (20 mL, 0.05M_{Co}) and heated to 100° C. Tri(octyl)phosphine (3.0 mmol) was added, and the solution was heated to 160° C. 1.0M Super-Hydride in THF (2.0 mL, 2.0 mmol) was added and the solution was stirred vigorously for 20 minutes. Additional Super-Hydride was added (1.0 mL, 1.0 mmol), and the solution was stirred for 20 more minutes. The reaction was then slowly cooled to room temperature and then removed from the nitrogen atmosphere. The nanoparticles were purified by centrifugation in pure ethanol (approximately 3 mg/mL) for 2 hours at 6000 g followed by decantation, two times, and then air-dried.

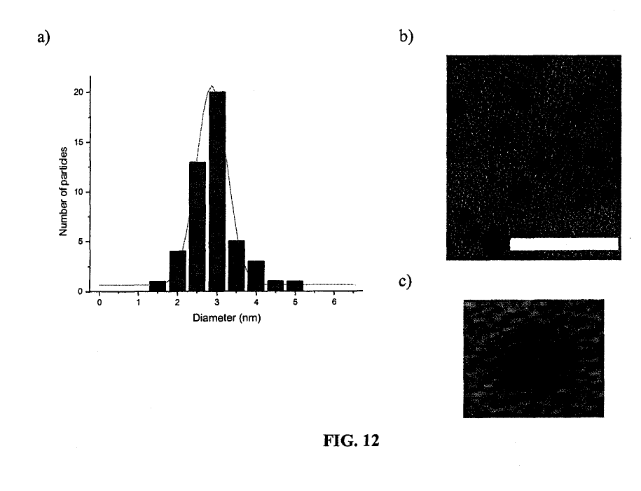

FIG. 12a shows the particle size distribution as synthesized determined from high-resolution TEM micrographs. The average nanoparticle size was 2.8 nm (σ=0.7). FIG. 12b shows a field of view with scale bar=10 nm. FIG. 12 shows a typical 4 nm nanoparticle showing that the nanoparticles are single-crystalline. FTIR analysis shows that the ligands are bound to the nanoparticle surface by carboxylate groups only, leaving only amines as the reactive functional groups on the nanoparticle. The nanoparticles were soluble in almost all polar solvents, except for acetonitrile. However, they easily formed aggregates because of the magnetic dipole and hydrogen-bonding interactions between them. The solubility was greatly increased by the addition of a strong acid like 1M HCl.

Covalent Linkage of Cobalt Nanoparticle to DNA Hairpin-Loop:

The DNA hairpin-loop (“stem-loop” or “molecular beacon”) was synthesized by Genset Oligos. The terminal nucleotides made up a 7-base-pair stem, with a 24-base-pair loop. The fluorescein dye, FAM, was appended at the 5′ end, and the quencher, Dabcyl, at the 3′ end. The eighteenth base, denoted as T′, was a thymine with an internally modified free amine. The sequence of the oligonucleotide is (5′→3′) given below with the portion in parenthesis representing the “loop” of the hairpin-loop structure:

FAM-GCGCCCT-(AAACTGGTGGT′GGAATGCGTCATG)-AGGGCGC-
Dab

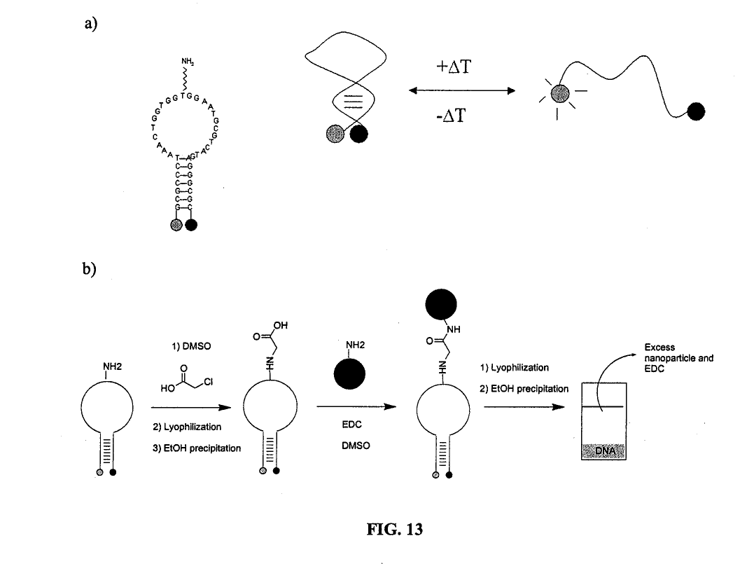

Oligonucleotide dehybridization causes an increase in fluorescence, as the efficiency of Dabcyl quenching of FAM fluorescence is distant dependent. The idealized structure of the hairpin-loop and the scenario of increased fluorescence upon dehybridization are shown below in FIGS. 13a and b.

Upon receiving the oligonucleotides, a 30 μm stock solution was made in DMSO. To obtain carboxylic acid functionalized hairpin-loops, 100 μL of the stock solution (3 nmol) was shaken overnight with 100 μL chloroacetic acid in DMSO (300 μM) (10×, 30 nmol) at room temperature, using standard nitrogen techniques. The oligonucleotide was purified by overnight ethanol precipitation and then redispersed in 500 μL DMSO. A tenfold excess of cobalt nanoparticle (3 mg, approximately 30 nmol) and a large excess of ethyldiethylaminopropylcarbodiimide (EDC) were added and the solution was shaken overnight. The sample was lyophilized, and then purified by overnight ethanol precipitation. The DNA/nanoparticle assembly was recast in 1×PBS buffer to make a 1 μM solution. The coupling scheme is shown in FIG. 13c.

Application of RFMF:

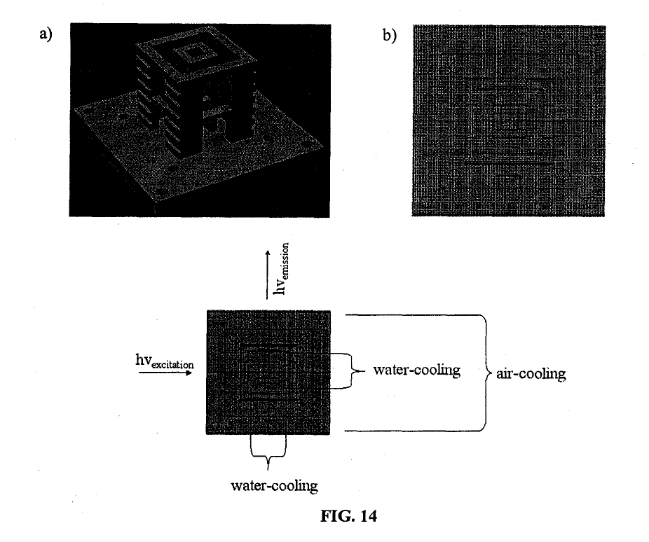

In order to apply RFMF in the fluoremeter with good global temperature control, a customized cell holder (FIG. 14) was constructed to replace the standard cell holder in the spectrophotometer. The cell holder was designed using Rhinoceros 3D modeling software and printed with ABS plastic using a Stratsys 3-D printer. The solenoid (Cu wire, 7 turns, d=3 cm, h=3 cm) was potted using epoxy and painted black to eliminate fluorescence from the polymer. A 0.5 cm gap was placed between the solenoid and the cuvette. Water-cooling was provided between the solenoid and sample on the two sides that were not in the light path of fluoremeter to limit radiative heat transfer from the solenoid; the cooled water (15° C.) was pumped through Master-Flex tygon tubing with a peristaltic pump, and the top portion of the cuvette that was not in the solenoid core was wrapped with tubing. The entire apparatus was cooled with a small cooling fan (Nidec Gamma 28).

RF was applied with a HP8648C signal generator coupled with a linear amplifier (+35 dBm, 1 mW input saturation, 4 W output saturation). The signal generator was driven at maximum output (+14.5 dBm) in order to ensure a 4 W signal from the amplifier; the 4 W output after the amplifier was determined by use of a bi-directional coupler (e-meca) and spectrum analyzer (Agilent E4407B). The calculated field strength was 8.2 mT.

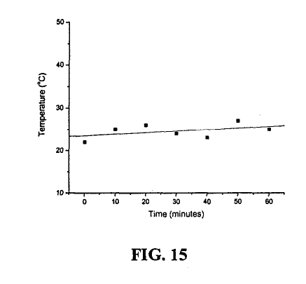

FIG. 15 shows the global solution temperature of 1×PBS buffered water under continuous RFMF irradiation at 300 MHz, given the conditions described above. The temperature is held within ±4° C. of room temperature as measured by a thermocouple (Omega).

Spectroscopy:

Fluorescence spectra were averaged four times with, λ_{exc}=495 nm. Fluorescence spectra were obtained using a Jovin-Ybon Spex Fluoromax-3 spectrophotometer (3 mm×3 mm quartz cuvette, 1 nm slits, 0.5 s integration).

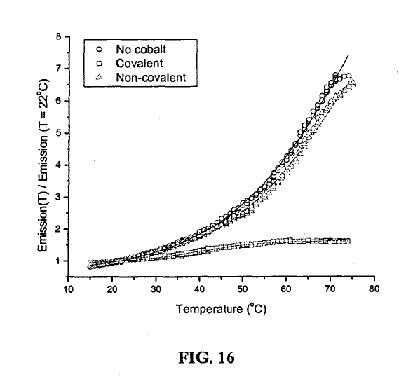

FIG. 16 shows the melting curves of the hairpin-loop (as described in Example 1) without cobalt nanoparticle antennae, hairpin-loop with an equimolar amount of non-covalently attached cobalt nanoparticle antennae, and hairpin-loop with covalently attached nanoparticle antennae. The melting point is lowered by induced mechanical stress or steric hindrance from the nanoparticle. Also, the fluorescence is greatly quenched by coupling with nanoparticle surface plasmons. To account for the effects of destabilization and fluorescence quenching by covalently attached nanoparticles, effective temperatures obtained from fluorescence data are calculated from the melting curves of each individual sample. Therefore, the samples are self-consistent.

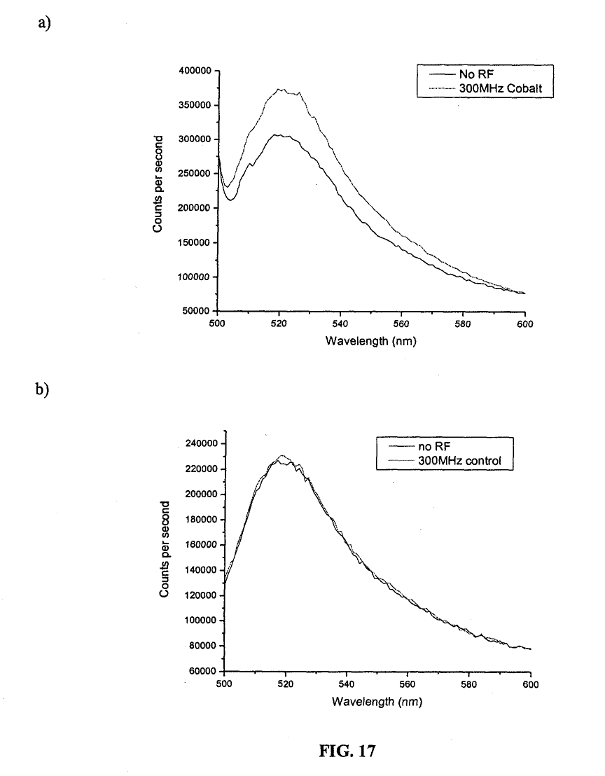

FIG. 17 shows the fluorescence spectra of FAM emission in a 300 MHz field. The increased intensity of FAM emission in FIG. 17a is evidence of selective dehybridization of the hairpin-loop with covalently attached nanoparticle antennae at 300 MHz. FIG. 17b shows the emission spectrum from hairpin-loop without any cobalt nanoparticle antennae. No effective temperature change due to RFMF is observable without nanoparticle antennae. The effective temperature change due to RFMF in FIG. 17a is ΔT=+18° C.

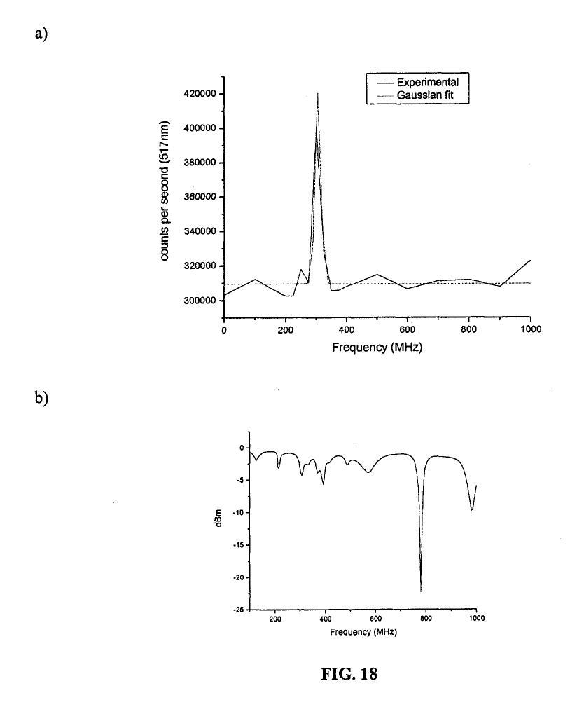

FIG. 18a shows the frequency response of the hairpin-loop with covalently attached nanoparticle antennae from 100 MHz-1 GHz, measured in 25 MHz steps; y-axis data points are the fluorescence emission values at 517 nm taken from spectra obtained under the same conditions as in FIG. 17. The frequency dependent selective dehybridization of the DNA hairpin-loop was not related to the field strength at that frequency, as can be seen by the reflected power spectrum of the coil from 100 MHz to 1 GHz in FIG. 18b, as measured by an Agilent 8417E RF network analyzer. The response is resonant, with a Gaussian fit showing a full-width-half-maximum (FWHM) of approximately 40 MHz.

### Example 2

### Selective Photocleavage and Michael Addition Induced by UV Radiation

This example demonstrates a possible mechanism for the selective photocleavage of covalent bonds for a shuttle covalently bound between molecular subunits. Specifically, the example provides methods and compositions for controlling relative Michael reactant/product equilibrium by UV irradiation.

### Synthesis of 5-(p-tolyl)-5-cyano-4,4-dimethyl-2-pentanone (X) and 4-(p-tolyl)-4-cyano-3,3-dimethyl-N,N-dimethylbutaneamide (Y)

All reagents were of the highest purity available and were used as received from Sigma-Aldrich unless otherwise indicated. Reactions under nitrogen were performed using standard techniques. ¹H-NMR spectra were taken on a Varian Unity 300 MHz NMR spectrometer in CDCl₃. FTIR was taken using a Digi-Lab Excalibur FTS 3000 (neat, 64 scans, 4 cm⁻¹ resolution).

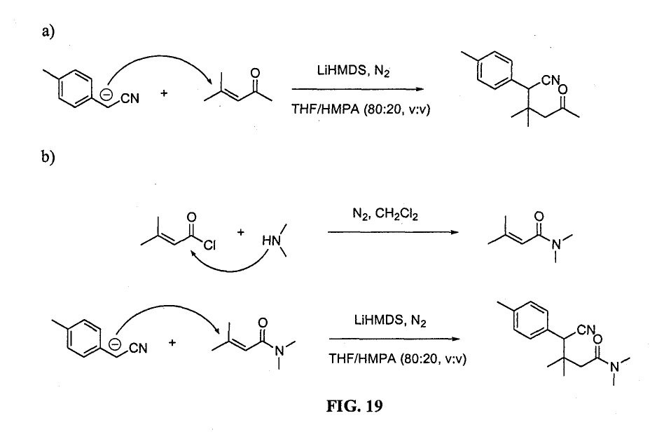

The synthetic schemes for (X) and (Y) are provided in FIGS. 19a and b, respectively.

### Synthesis of 5-(p-tolyl)-5-cyano-4,4-dimethyl-2-pentanone (X)

660 μL p-tolyl-acetonitrile (5 mmol) and 572 μL mesityl oxide were stirred in 50 mL THF/HMPA (v:v 80:20) for 30 minutes at −78° C. under nitrogen. 5 mL of 1M LiHMDS in THF (5 mmol) was added dropwise. The reaction was brought slowly to room temperature over 90 minutes, and stirred for two days at room temperature. The solution was extracted using ether/1M HCl, and purified by silica gel flash chromatography (20:80 ethyl acetate/hexanes, v:v). After solvent removal by rotary evaporation, the sample was dried under high vacuum overnight. ¹H-NMR δ (ppm): 7.45-7.25 (4H, m), 4.82 (1H, s), 2.98 (3H, s), 2.36 (3H, s), 2.44-2.11 (2H, AB quartet), 1.25 (3H, s), 1.08 (3H, s). FTIR 2236 (m, s, CN), 1712.1 (s, br, CO).

### Synthesis of 4-(p-tolyl)-4-cyano-3,3-dimethyl-N,N-dimethylbutaneamide (Y)

557 μL 3,3-dimethylacryloyl chloride (5 mmol) and 6.25 mL 2M dimethylamine in THF (2.5×, 12.5 mmol) were stirred under nitrogen at 0° C. for 30 minutes in 43.25 mL CHCl₂. The compound was purified by extraction with ether/1M HCl. Ether was removed by rotary evaporation to yield 3-methyl-N,N-dimethyl-2-buteneamide.

5 mL of 1M LiHMDS in THF (5 mmol) was added dropwise to a solution of 660 μL p-tolyl-acetonitrile (5mmol) in 25 mL THF/HMPA (80:20, v:v), and stirred for 30 minutes at −78° C. under nitrogen. 25 mL of 0.2M 3-methyl-N,N-dimethyl-2-buteneamide in THF/HMPA was added dropwise. The reaction was brought slowly to room temperature over 90 minutes, and stirred for two days at room temperature. The solution was extracted using ether/1M HCl, and purified by silica gel flash chromatography (70:30 ethyl acetate/hexanes, v:v). After solvent removal by rotary evaporation, the sample was dried under high vacuum overnight. ¹H-NMR: δ 7.26-7.12 (4H, m), 4.82 (1H, s), 2.97 (6H, s), 2.34 (3H, s), 2.43-2.11 (2H, AB quartet), 1.23 (3H, s), 1.05 (3H, s). FTIR (cm⁻¹) 2235 (m, s, CN), 1643 (s, br, CONR₂).

Control of Michael Reaction Equilibrium by Wavelength Selective Photocleavage Reactions:

Photocleavage reactions were performed under nitrogen in a photochemical micro-reaction assembly (Ace Glass model 7880) in cyclohexane (1 mM) using a Pen-Rey 5.5W low-pressure mercury UV-lamp. (X) was irradiated with λ>290 nm by filtering lower wavelengths with borosilicate glass. (Y) was irradiated with λ˜215 nm by filtering with a 3:1:0.5 (v:v) mixture of ethanol:water:acetone with constant bubbling of nitrogen through the solution. HPLC of (X) and (Y) were performed on a normal-phase silica column with 70:30 and 50:50 (v:v) heptane/THF as eluent, respectively. HPLC chromatographs were obtained on a Varian Pro Star HPLC (monitored by UV absorption at 254 nm).

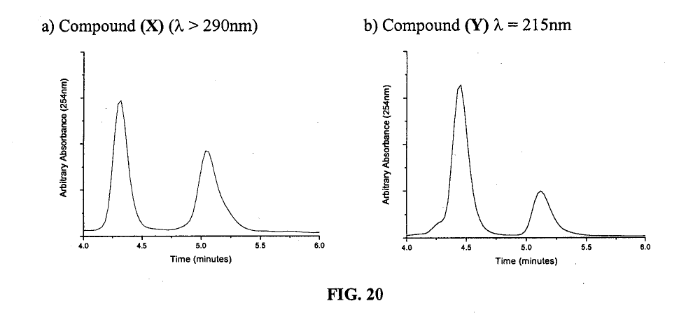

FIGS. 20a and b show the respective HPLC chromatographs of (X) and (Y) after 48 hours UV irradiation at λ>290 nm and λ=215 nm, respectively. In each chromatograph, the left peak represents the cleavage product/Michael acceptor, 3-(p-tolyl)-3-cyano-2-methyl-2-butene (Z), and the right peak represents the initial molecules (X) and (Y)/Michael products. The Michael donors, acetone and N,N-dimethylacetamide, are not present in the spectra, as they are removed by water/heptane extraction during HPLC sample preparation. The cleavage reactions are wavelength selective as determined by thin-layer chromatography (TLC) analysis. The cleavage reaction followed a Norrish Type II pathway, and not a Type I cyclization, as determined by NMR analysis of products purified by silica-gel flash chromatography—(Z) ¹H-NMR δ (ppm): 7.33-7.17 (4H, m), 2.38 (3H, s), 2.25 (3H, s), 1.92 (3H, s).

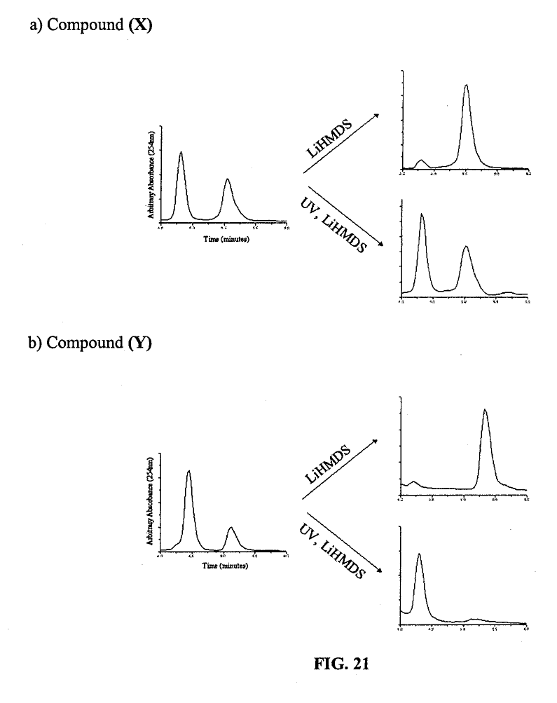

After 48 hours, the sample was aliquoted into two samples by removing half the sample volume from each reaction mixture, and an equimolar amount of LiHMDS was added to each sample. One aliquot was allowed to react in the presence of UV irradiation, and another was stirred under nitrogen in the dark at room temperature. After 48 hours, the relative equilibrium of the Michael reaction between (Z) and the acetone enolate or the N,N-dimethylacetamide amidine was controlled by the UV irradiation, as shown in FIGS. 21a and b, respectively.

It is understood that the invention is not confined to the particular embodiments set forth herein as illustrative, but embraces all such forms thereof as come within the scope of the following claims.

What is claimed is:

1. A molecular shuttle device comprising:
(a) two or more molecular subunits connected to form a molecular chain, the subunits defining binding positions along the molecular chain; and
(b) a shuttle capable of binding at each of the binding positions;
wherein the shuttle moves between binding positions in response to one or more input signals which interact with the molecular subunits.

2. The molecular shuttle device of claim 1, wherein the molecular chain and the shuttle are in solution.

3. The molecular shuttle device of claim 1, wherein interactions between the one or more input signals and the subunits break bonds between the shuttle and the subunits.

4. The molecular shuttle device of claim 1, wherein interactions between the one or more input signals and the subunits make bonds between the shuttle and the subunits.

5. The molecular shuttle device of claim 1, wherein the input signals for neighboring molecular subunits are resolvable.

6. The molecular shuttle device of claim 1, wherein the shuttle is capable of binding at the binding positions in the absence of an input signal.

7. The molecular shuttle device of claim 1, wherein the shuttle does not bind to the binding positions in the absence of an input signal.

8. The molecular shuttle device of claim 1, further comprising an antenna attached to each molecular subunit for receiving an input signal, wherein the antennae for neighboring molecular subunits are adapted to receive resolvable input signals.

9. The molecular shuttle device of claim 8, wherein the antennae comprise molecules independently selected from the group consisting of nanoparticles, single-molecule magnets, and dye molecules.

10. The molecular shuttle device of claim 1, wherein the molecular subunits define only two binding positions.

11. The molecular shuttle device of claim 10, further comprising a fluorophore and a quencher for the fluorophore, wherein one of the fluorophore or the quencher is attached to the shuttle and the other of the fluorophore or the quencher is attached to a molecular subunit.

12. The molecular shuttle device of claim 1, wherein the molecular chain comprises three molecular subunits and further wherein the input signal for each of the three molecular subunits is resolvable with respect to the input signals of the other two molecular subunits.

13. The molecular shuttle device of claim 12, wherein the molecular chain comprises a repeating pattern of the three molecular subunits.

14. The molecular shuttle device of claim 1, wherein the shuttle is capable of binding to the molecular subunits individually.

15. The molecular shuttle device of claim 1, wherein the shuttle is capable of binding between adjacent pairs of molecular subunits.

16. The molecular shuttle device of claim 1, wherein the molecular subunits and the shuttle comprise oligonucleotide sequences.

17. The molecular shuttle device of claim 1, wherein the molecular subunits comprise molecules capable of forming zwitterions in response to an input signal and the shuttle comprises a charged molecule capable of undergoing ionic bonding to the zwitterions.

18. The molecular shuttle device of claim 1, wherein the molecular subunits comprise chemical moieties and the shuttle comprises functional groups capable of forming photocleavable covalent bonds with the chemical moieties of the subunits.

19. The molecular shuttle device of claim 1, wherein the input signals are independently selected from the group consisting of magnetic inputs and electric inputs.

20. The molecular shuttle device of claim 1, wherein the input signals comprise compounds that change the chemical environment of the device.

21. The molecular shuttle device of claim 1, wherein the molecular chain is attached to a solid support.

22. The molecular shuttle device of claim 1, wherein the one or more input signals comprise an input signal sequence that interacts with the molecular chain and the shuttle moves between binding positions in response to the input sequence.

23. The molecular shuttle device of claim 22, wherein the molecular subunits and the shuttle are in solution.

24. The molecular shuttle device of claim 22, wherein interactions between the input signal sequence and the subunits break bonds between the shuttle and the subunits.

25. The molecular shuttle device of claim 22, wherein interactions between the input signal sequence and the subunits make bonds between the shuttle and the subunits.

26. The molecular shuttle device of claim 22, wherein the input signals for neighboring molecular subunits are resolvable.

27. The molecular shuttle device of claim 22, further comprising an antenna attached to each molecular subunit for receiving an input signal, wherein the antenna for neighboring molecular subunits are adapted to receive resolvable input signals.

28. The molecular shuttle device of claim 22, wherein the molecular subunits define two binding positions on the molecular chain and further wherein the shuttle moves between the two binding positions in response to an input signal sequence, to provide a molecular switch.

29. The molecular shuttle device of claim 28, further comprising a fluorophore and a quencher, wherein one of the fluorophore or the quencher is attached to the shuttle and the other of the fluorophore or the quencher is attached to a molecular subunit, such that the fluorophore is quenched when the shuttle is in one of the two binding positions.

30. The molecular shuttle device of claim 22, wherein the molecular subunits define at least three binding positions on the molecular chain and further wherein the shuttle moves between the at least three binding positions in response to an input signal sequence to provide a molecular assembly line.

31. The molecular shuttle device of claim 30, wherein the shuttle moves bi-directionally between the binding positions.

32. The molecular shuttle device of claim 23, wherein the molecular subunits comprise biomolecules, the shuttle comprises a biomolecule capable of hybridizing to the molecular subunit biomolecules, and the input signals interact with the molecular subunits to selectively dehybridize the shuttle from the molecular subunits.

33. The molecular shuttle device of claim 23, wherein the molecular subunits comprise biomolecules, the shuttle comprises a biomolecule capable of selectively binding to the molecular subunit biomolecules, and the input signals interact with the molecular subunits to selectively activate binding interactions between the shuttle and the molecular subunits.

34. The molecular shuttle device of claim 22, wherein the molecular subunits comprise biomolecules, the shuttle comprises a biomolecule capable of selectively binding to the molecular subunit biomolecules, and the input signals produce changes in the pH of the system to selectively activate binding interactions between the shuttle and the molecular subunits.

35. The molecular shuttle device of claim 22, wherein the molecular subunits comprise molecules capable of forming zwitterions, the shuttle comprises a charged molecule capable of undergoing ionic bonding to the zwitterions, and the input signals selectively convert the molecular subunit molecules from non-zwitterionic to zwitterionic forms.

36. The molecular shuttle device of claim 22, wherein the molecular subunits comprise molecules capable of undergoing a conformational change from a first conformation to a second conformation, the shuttle comprises a molecule capable of binding to the molecular subunit molecules in either their first or their second conformation, and the input signals selectively convert the molecular subunit molecules from one conformation to the other.

37. The molecular shuttle device of claim 22, wherein the molecular subunits comprise chemical moieties, the shuttle comprises functional groups capable of covalently bonding with the chemical moieties of the subunit molecules, and the input signals photocleave the covalent bonds between the molecular subunit molecules and the shuttle.
